SEQUENCE LISTING (20 sequences)

SEQ ID NO: 1 - protein, 62 aa, Artemia Sp.
ANDCKCPNGCSCPNCANGGCQCGDKCECKKQSCHGCGEQCKCGSHGSSCHGSCGCGDKCECK

SEQ ID NO: 2 - protein, 154 aa, Artificial Sequence
  SUMO-MT fusion protein
HHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGMDCCKNGCTCAPNCKCAKDCKCCKGCECKSNPECKCEKNCSCNSCGCH

SEQ ID NO: 3 - DNA, 34 bp, Artificial Sequence
  MT-Not I primer
acctatgcggccgcaaatggactgctgcaagaac

SEQ ID NO: 4 - DNA, 30 bp, Artificial Sequence
  dT-Spe I primer 1
gcaccaactagtgccttttttttttttttt

SEQ ID NO: 5 - DNA, 30 bp, Artificial Sequence; identical to SEQ ID NO: 4
  dT-Spe I primer 2

SEQ ID NO: 6 - DNA, 30 bp, Artificial Sequence; identical to SEQ ID NO: 4
  dT-Spe I primer 3

SEQ ID NO: 7 - DNA, 33 bp, Artificial Sequence
  MT Nco I primer
gctacacatatgtccatggactgctgcaagaac

SEQ ID NO: 8 - DNA, 30 bp, Artificial Sequence
  MT Sal I primer
acgaacgtcgacgccttttttttttttttt

SEQ ID NO: 9 - protein, 61 aa, Oryctolagus cuniculus
MDPNCSCATRDSCACASSCKCKECKCTSCKKSCCSCCPAGCTKCAQGCICKGALDKCSCCA

SEQ ID NO: 10 - protein, 61 aa, Cercopithecus aethiops
MDPNCSCATGVSCTCADSCKCKECKCTSCKKSCCSCCPVGCAKCAQGCVCKGASEKCNCCA

SEQ ID NO: 11 - protein, 61 aa, Homo sapiens
MDPNCSCAAGDSCTCAGSCKCKECKCTSCKKSCCSCCPVGCAKCAQGCICKGASDKCSCCA

SEQ ID NO: 12 - protein, 60 aa, Ictalurus punctatus
MDPCECSKTGTCNCGTSCKCSNCQCACCKKSCCSCCPSGCSKCASGCVCKGDTCDSKCCQ

SEQ ID NO: 13 - protein, 62 aa, Xenopus laevis
MDPQDCKCETGASCSCGTTCSCSNCKCTSCKKSCCSCCPAECSKCSQGCHCEKGSKKCSCCN

SEQ ID NO: 14 - protein, 71 aa, Mytilus edulis
PGPCNCIETNVCICGTGCSGKCCRCGDACKCASGCGCSGCKVVCKCSGTCKCGCDCTGPTNCKCESGCSCK

SEQ ID NO: 15 - protein, 68 aa, Lytechinus pictus
MPGPDVKCFCCRDGKECACGGGECCITGKCCKEGDRTCCGKCSNAACKCADGCKCEGACACTMGNCTC

SEQ ID NO: 16 - protein, 43 aa, Drosophila melanogaster
MVCKGCGTNCQCSAQKCGDNCACNKDCQCVCKNGPKDQCCSNK

SEQ ID NO: 17 - protein, 62 aa, Caenorhabditis elegans
VCKCDCKNQNCSCNTGTKDCDCSDAKCCEQYCCPTASEKKCCKSGCAGGCKCANCECAQAAH

SEQ ID NO: 18 - protein, 74 aa, Oryza sativa
MSCSCGSSCSCGSNCSCGKKYPDLEEKSSSTKATVVLGVAPEKKAQQFEAAAESGETAHGCSCGSSCRCNPCNC

SEQ ID NO: 19 - protein, 75 aa, Triticum aestivum
MSCNCGSGCSCGSDCKCGKMYPDLTEQGSAAAQVAAVVVLGVAPENKAGQFEVAAGQSGEGCSCGDNCKCNPCNC

SEQ ID NO: 20 - protein, 62 aa, Candida glabrata; identical to SEQ ID NO: 1

(19) United States
(12) Patent Application Publication
(10) Pub. No.: US 2020/0254422 A1
(43) Pub. Date: Aug. 13, 2020

(54) Compositions and Methods for Removing Heavy Metals from Contaminated Materials

(71) Applicant: MGP Biotechnologies, Inc., Irvine, CA (US)

(72) Inventors: Roger A. Acey, Long Beach, CA (US); Gwen R. Jordaan, Irvine, CA (US)

(21) Appl. No.: 15/741,125

(22) PCT Filed: Jul. 1, 2016
(86) PCT No.: PCT/US2016/040846

§ 371 Date: Apr. 27, 2018

Related U.S. Application Data
(60) Provisional application No. 62/188,416, filed on Jul. 2, 2015.
(60) Provisional application No. 62/188,421, filed on Jul. 2, 2015.
(60) Provisional application No. 62/301,290, filed on Feb. 29, 2016.

(51) Int. Cl. B01J 20/24; B01J 20/26; B01J 20/28; B01J 20/34; C07K 14/825; B01D 15/20; B01D 15/38; B01D 11/04; B01D 69/14; B01D 69/02; C02F 1/28; C02F 1/44; C22B 3/20; C22B 7/00

(52) U.S. Cl. CPC B01J 20/24; C02F 2101/20; B01J 20/28038; B01J 20/3475; B01J 20/3425; C07K 14/825; B01D 15/203; B01D 15/3828; B01D 11/0492; B01D 69/144; B01D 69/147; B01D 69/02; C02F 1/286; C02F 1/288; C02F 1/44; C22B 3/20; C22B 7/006; B01D 2325/12; B01J 20/26

(57) ABSTRACT

Metal-binding proteins, such as metallothionein proteins, are disclosed for removing metals from substrates in need of having such metals removed therefrom. Specifically, metallothionein proteins according to SEQ ID NO:1, 2, or 9-20 are disclosed for removing metals from liquid substrates. Associated methods for removing metals from substrates using metallothionein proteins are also disclosed.

### CROSS-REFERENCE TO RELATED APPLICATIONS

The present application claims the benefit under 35 U.S.C. § 119(e) to U.S. Provisional Patent Applications 62/188,416 filed Jul. 2, 2015, 62/188,421 filed Jul. 2, 2015, and 62/301,290 filed Feb. 29, 2016. Each of these prior applications is incorporated by reference herein in their entirety.

### FIELD

The present disclosure relates to compositions and methods for removing heavy metals from contaminated samples. More specifically the present disclosure relates to removing heavy metals from contaminated samples using metallothionein proteins and to compositions and methods for removing heavy metals from organic solvents.

### BACKGROUND

Metal recovery and metal remediation and the associated need for efficient and safe methods for clean up of metal waste is a continuing environmental and business concern due to the toxicity and potential risk to human health posed by metal contaminants, as well as the economic value of precious heavy metals. Indeed, as the discharge of toxic wastes from agricultural, industrial, and other commercial operations continues, the need for effective, safe, and low-cost metal remediation methods increases. In a recent report by the United States Environmental Protection Agency (US EPA), metal contamination remains and historically has been a key concern at many contaminated sites. In addition, there are numerous published reports of damage to wildlife, livestock, plant life as well as danger to human health as a result of metal poisoning from contaminated soil or waste matter. For example, a primary concern to humans is the health hazard created by lead (Pb) contamination. Exposure to lead can occur through a variety of methods such as by ingestion of lead from food, water, soil, or even inhalation of dust. Lead poisoning is extremely dangerous and potentially fatal, with symptoms including seizures, mental retardation, and behavioral disorders. Therefore, methods for metal remediation are extremely valuable both for their protection of our environment as well as for protection from diseases.

Recovered metals from various waste, discard or recycling efforts provide immense economic value as well as augmenting environmental pollution control. Metal recovery can be from innumerable and varied sources such as from waste electronic devices (e.g., transistors, chips, transformers, bus bars, cathodes, microprocessors, populated computer circuit boards PCBs, motherboards, etc.). Costs associated with hazardous disposal of industrial waste in the absence of metal reclamation are enormous. Therefore, metal recycling or reuse of metal extracted from scrap or discarded metal-containing items not only reduces the volume and cost of metal waste requiring specialized disposal and handling efforts, but the reclaimed metal can also be resold or reused to provide additional economic value.

Prior art attempts at treating metal contamination have traditionally employed cleanup technologies which consist primarily of physically removing and then disposing of contaminated matter. These methodologies are not only labor intensive and less efficient, but also carry a high expense associated with removal and disposal of large or bulk quantities of contaminated waste. Metal contamination is especially difficult to remediate because, unlike other types of waste such as chemical or organic matter, metals cannot be directly destroyed or converted. For example, current technologies for remediating metal contaminated soils consist primarily of landfilling or soil excavation with physical or chemical separation of the metal contaminants. Treatment of contaminated ground water usually involves flushing, filtration, or chemical extraction to remove the contaminating metals. As a result, the cost of soil or ground water remediation is high, ranging in the hundreds of thousands to millions of dollars in projected five-year costs per site.

In addition, the risk to humans and the environment from heavy metal contamination is not limited to soil or ground water, but also includes other sources such as industrial waste, sludge waste, wastewater, radioactive waste (such as radionuclides from research and medical waste), and mining waste. Depending on the physical and chemical form of the metal contaminant to be removed, as well as the cost-benefit analysis for a particular remediation approach, which of the existing technologies is better suited for a particular site will vary. However, due to the high cost of traditional cleanup technologies, there still remains a great need for a less-expensive, safe and effective heavy metal recovery and cleanup technology.

There are some technologies currently available for the recovery or remediation of heavy metal contaminated waste. In general, these technologies combine one or more of the following general approaches: isolation, immobilization, toxicity reduction, physical separation, or extraction of metal contamination from a waste product. Isolation technologies utilize a containment strategy in an attempt to confine a contaminated site or area so as to prevent further spread of the toxic metal waste. Immobilization technologies reduce the mobility of metal contaminants and include systems which provide an impermeable barrier to separate underlying layers of soil (containing the metal contaminants) from the topsoil layer. Also used are physical barriers which restrict the flow of uncontaminated groundwater through a contaminated site. Additionally, there are toxicity reduction processes which generally use chemical or biological techniques to decrease the toxicity or mobility of metal contaminants. Included in toxicity reduction processes are biological treatment technologies, which apply newer biotechnical approaches.

Metal remediation is a relatively new application of biological treatment technologies and includes processes such as bioaccumulation, phytoremediation, phyotextraction, and rhizofiltration. All of these biological treatments use certain plants and microorganisms to remediate metals through either adsorption, absorption, or concentration of contaminating metal ions. For example, in bioaccumulation, plants or microorganisms actively take up and accumulate metals from contaminated surroundings.

In phytoremediation, specific plants that have developed the ability to selectively remove metal ions from soil are used. Such plants include certain “hyperaccumulator” species such as the alpine pennycrass plant, which is capable of accumulating metals at levels of 260 times greater than most plants before showing toxicity symptoms. Most hyperaccumulator plants, however, are very slow growing and have specific growth requirements. Some of these growth requirements are not conducive to the use of these plants at sites or in situations where metal recovery or remediation is needed. Furthermore, there are very few plant species known or available for recovery or remediation use. Therefore, given the persistent and high incidence of metal contamination at environmental and waste sites (about 75% of Superfund Sites contain metal ions as a form of contamination, U.S. EPA, 1996), more efficient methods and approaches for removing heavy metals from contaminated sources are still needed.

More recently, in an attempt to meet these needs, biotechnological approaches have been employed as an alternative strategy to metal recovery and remediation. Included in these biotechnology approaches are the use of tobacco plants that have been manipulated to express metallothionein genes. Metallothioneins (MTs) are small metal-binding proteins ubiquitously distributed throughout the animal kingdom. They have high metal-binding affinities and are believed to be important in controlling the intracellular levels of free metal ions. However, little else is known about their function or biological purpose. Metallothioneins were first discovered in 1957 in horse tissue. Since then, they have been identified in species ranging from fungi and shellfish to mice and humans.

The structural features of MTs include a high cysteine composition and lack of aromatic amino acids. The cysteine residues are responsible for the protein's high affinity metal ion binding capabilities. In general, MTs have a high degree of amino acid sequence similarity. However, the proteins or known gene sequences encoding the proteins have been used primarily in either the research setting or in disease treatment methodologies.

Accordingly, one of the objects of the present methods and compositions is to provide novel metal-binding proteins for the removal of metals from a variety of substrates. This technology would allow for the efficient, cost effective, safe and simple removal of heavy metals from environmental waste or other materials contaminated with heavy metal.

### SUMMARY

The recombinant chimeric metal-binding proteins disclosed herein are capable of high capacity and high affinity metal-binding, making them particularly suitable for use in pollution control, metal recycling, metal mining and other metal recovery and metal remediation technologies.

These and other objectives are achieved by the compositions and methods disclosed herein which provide for the efficient and reliable sequestration of heavy metals from a variety of sources using a regenerative metal-binding material comprised of at least one metal-binding protein. The metal-binding proteins can be expressed and produced easily for all purposes where binding of one or more heavy metals is desired.

Disclosed herein are recombinant chimeric metallothionein (MT) proteins having a sequence at least 90% identical to full-length SEQ ID NO:1.

Also disclosed herein is a device for removing heavy metals from a substrate comprising a regenerative metal-binding material comprising an MT protein having a sequence at least 90% identical to one of SEQ ID Nos. 1, 2, and 9-20; wherein the regenerative metal-binding material binds heavy metals thereby removing the heavy metals from a substrate; and wherein the binding of heavy metal to the regenerative metal-binding material is reversible.

Also disclosed herein is a method for removing metals from a substrate comprising contacting a substrate having heavy metals therein with a regenerative metal-binding material comprising a MT protein having a sequence at least 90% identical to one of SEQ ID NO:1, 2, and 9-20; binding the heavy metal to the regenerative metal-binding material thereby producing a substrate having less heavy metal contained therein.

In some embodiments, the regenerative metal-binding material comprises the MT protein in an aqueous solution. In some embodiments, the regenerative metal-binding material comprises the MT protein associated with a solid support. In some embodiments, the regenerative binding material is reusable. In some embodiments, the solid support is a resin. In some embodiments, the solid support is a membrane.

In some embodiments disclosed herein, the substrate is a liquid. In some embodiments, the substrate is a metal-containing organic liquid. In certain embodiments, the heavy metal is a heavy metal complex.

In some embodiments, the method further comprises releasing the bound heavy metal from the regenerative metal-binding material; and regenerating the metal-binding capacity of the regenerative metal-binding material.

In some embodiments, the metal is substantially released from the metal-binding protein at a pH of about 1. In some embodiments, the metal-binding protein has a substantial metal-binding affinity at a pH above about pH 5.

Also disclosed herein is an aqueous solution for extracting a metal from an organic solvent, the aqueous solution comprising a metal-binding protein having an amino acid sequence at least 90% identical to one of SEQ ID NO:1, 2, and 9-20.

In some embodiments, the metal is a heavy metal, a radioactive metal, or a precious metal. In some embodiments, the metal is a transition metal. In some embodiments, the metal has an atomic weight greater than 40 g/mol.

In some embodiments, the organic solvent comprises dichloromethane, chloroform, diethyl ether, or hexane.

### BRIEF DESCRIPTION OF THE DRAWINGS

FIG. 1 depicts sequences of metallothionein proteins.

FIG. 2 depicts an elution profile of exemplary metal-binding proteins illustrating co-elution of metal-binding proteins with the heavy metal zinc.

FIG. 3 illustrates MT protein selectively binding heavy metals in solution.

FIG. 4 illustrates the removal of heavy metals from water.

FIG. 5 illustrates the recovery of removed metals from a membrane coated with MT proteins.

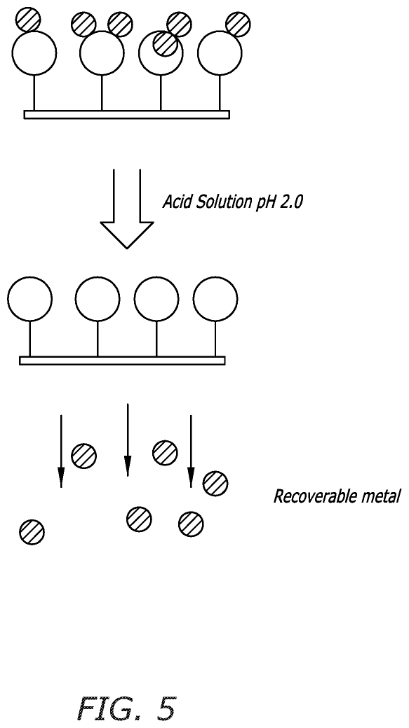

FIG. 6 illustrates the selectivity and affinity of MT proteins for binding heavy metals.

FIG. 7 depicts recombinant chimeric SUMO-MT purified from bacteria. Cells were collected and sonicated in 0.05 M phosphate/300 mM NaCl, buffer, pH 8.0. Half the extract was placed in a boiling water bath for ten min. Both samples were centrifuged and the supernatants incubated with NiNTA agarose. The chimeric protein was eluted with 400 mM histidine and analyzed by SDS PAGE (12% Bis Tris gel). Lane 1: boiled extract; Lane 2: non boiled extract; Lane M: markers with size indicated in kDa.

FIG. 8A-C illustrates three embodiments of a metal-containing medication or solution removal device in the form of syringe dosing devices.

FIG. 9A-D depicts a process of removing thimerosal from a vaccine. FIG. 9A depicts a syringe pre-loaded with magnetic beads coupled to MT. FIG. 9B depicts the syringe filled with vaccine. FIG. 9C depicts a circular magnet placed at the base of the syringe and slid toward the top of the syringe. FIG. 9D depicts beads captured by the magnet and thimerosal-free vaccine available for injection into a subject.

FIG. 10A-C depicts characterization of SUMO-MT. A typical elution profile using ¹⁰⁹Cd is shown in FIGS. 10A and 10C. The level of ¹⁰⁹Cd in each fraction was determined using liquid scintillation. The elution profile of the SUMO-MT without added metal was determined by applying the protein to the column and measuring the A₂₂₅ of the fractions (FIGS. 10B and 10C).

FIG. 11 depicts removal of metal (palladium nanoparticles) from an organic solution. A solution of palladium nanoparticles in chloroform with an aqueous solution of MT protein. The tube on the right is a control with palladium nano-particles remaining in the organic phase (lower layer) with an MT-containing aqueous buffer solution in the upper layer. The tube on the left depicts the palladium nanoparticles having left the lower organic phase and entered the MT-containing aqueous phase on top.

FIG. 12 depicts a metal-binding resin. A 0.1 mM solution of copper sulfate (colorless) was continuously passed through the resin column. Lane 1: MT bound to resin; Lane 2: After 15 min; Lane 3: After 60 min; Lane 4: 30 sec after addition of acid; Lane 5: 60 sec after addition of acid; Lane 6: Completely regenerated column.

FIG. 13A-B depicts the effect of pH on metal binding of recombinant SUMO-MT at pH 4.0 (FIG. 12A) and pH 10.0 (FIG. 12B).

### DETAILED DESCRIPTION

Metal-binding proteins such as metallothioneins (MTs) that have been isolated from various species such as humans, mice, bacteria species, crabs, fish, yeast and chickens, and are known to have very similar structural characteristics such as similar size (about 6.0-6.8 kDa), high amino acid sequence conservation, and a high percentage of cysteine residues in the proteins' total amino acid compositions. It is the cysteine composition of these MTs that accounts for the protein's binding affinity for heavy metals including transition metals, lanthanide metals, rare earth metals, actinide metals, and radioactive metals. The metal-binding proteins disclosed herein also bind heavy metals complexes in which the heavy metals are associated with a protein or another inorganic or organic molecule.

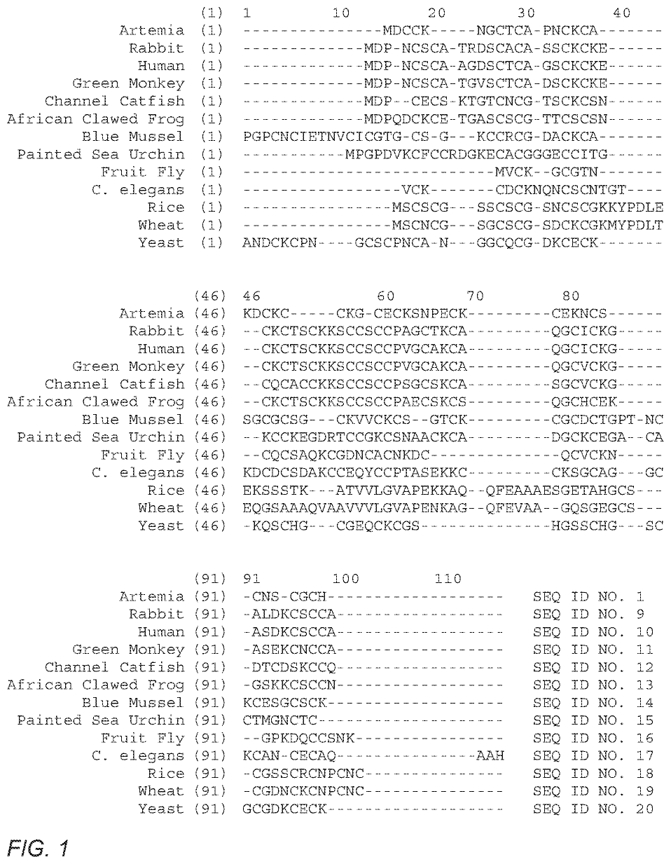

Species as divergent as humans and wheat express metallothionein proteins with similar binding affinities for heavy metals. These MT proteins contain from 12 to 22 cysteine residues, which are conserved across divergent species (FIG. 1). These cysteine residues form metal binding motifs responsible for the metal binding function of the proteins. One embodiment disclosed herein provides MT proteins immobilized on solid supports, such as membranes, wherein the MT are isolated from organisms including, but not limited to, mammals, fish, mollusks, echinoderms, crustaceans, reptiles, nematodes, grains and yeast. Non-limiting examples of these organisms include, but are not limited to, brine shrimp (Artemia, SEQ ID NO:1), rabbit (Oryctolagus cuniculus, SEQ ID NO:9), green monkey (Cercopithecus aethiops, SEQ ID NO:10), human (Homo sapiens, SEQ ID NO:11), channel catfish (Ictalurus punctatus, SEQ ID NO:12), African clawed frog (Xenopus laevis, SEQ ID NO:13), blue mussel (Mytilus edulis, SEQ ID NO:14), painted sea urchin (Lytechinus pictus, SEQ ID NO:15), fruit fly (Drosophila melanogaster, SEQ ID NO:16), roundworm (Caenorhabditis elegans, SEQ ID NO:17), rice (Oryza sativa, SEQ ID NO:18), wheat (Triticum aestivum, SEQ ID NO:19), and yeast (Candida glabrata, SEQ ID NO:20).

For example, the metal-binding proteins, and devices comprising them, called regenerative metal-binding supports, disclosed herein are useful in connection with the treatment of any substrate having a concentration of at least one metal, such as a heavy metal. As will be appreciated by those skilled in the art, such heavy metal containing substrates can be any environmental or industrial material such as ground water, drinking water, contaminated soil, waste, or the like, containing a concentration of metal. Similarly, the methods of the present disclosure are equally useful in treating industrial or municipal wastes containing metals that are desirable to remove. This broad utility makes the compositions and associated methods disclosed herein particularly useful in a wide variety of circumstances.

The metal-binding proteins and regenerative metal-binding materials disclosed herein are useful in the recovery of metals, particularly rare or precious metals from metal-containing substrates. The disclosed MT metal-binding proteins may bind to any metal, such as transition metals (e.g. arsenic, zinc, copper, cadmium, mercury, cobalt, lead, nickel, chromium, platinum, palladium, silver, gold, etc.), lanthanide, or rare earth metals, precious metal, actinide metals, or other radioactive metals (e.g., uranium, neptunium, plutonium, americium, curium, technetium, etc.).

In some embodiments the metal-binding protein may have a higher affinity for metals with an atomic weight greater than 40 g/mol, 50 g/mol, 70 g/mol, or 100 g/mol, than for metals with an atomic weight of less than 40 g/mol, 50 g/mol, 70 g/mol, or 100 g/mol.

In some embodiments, the metal may be any metal or metalloid that has a binding affinity to the MT protein, including, but not limited to, aluminum, scandium, titanium, vanadium, chromium, manganese, iron, cobalt, nickel, copper, zinc, gallium, germanium, arsenic, selenium, bromine, krypton, rubidium, strontium, yttrium, zirconium, niobium, molybdenum, technetium, ruthenium, rhodium, palladium, silver, cadmium, indium, tin, antimony, tellurium, iodine, xenon, cesium, barium, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold, mercury, thallium, lead, bismuth, polonium, astatine, radon, francium, radium, actinium, thorium, protactinium, uranium, neptunium, plutonium, americium, curium, berkelium, californium, einsteinium, fermium, mendelevium, nobelium, lawrencium, and rutherfordium. For example, the metal binding proteins disclosed herein may be useful in connection with the treatment of any organic solvent containing at least one metal (e.g., transition metal, precious metal, heavy metal, etc.). Similarly, the methods disclosed herein may be equally useful in treating industrial or municipal wastes containing metals that are desirable to remove. This broad utility makes the compositions and associated methods disclosed herein particularly useful in a wide variety of circumstances.

For example, the metal-binding proteins can be used in metal mining processes for the isolation and removal of precious metals such as gold, platinum, and silver. Doing so eliminates the need to use other toxic materials such as cyanide in the final stages of metal purification from ore. These same novel techniques can be utilized to recover such metals from industrial or municipal waste. With the ever-increasing use of disposable and other electronic devices, such waste sources are increasingly full of such metals, making recovery a worthwhile endeavor.

The metal-binding proteins disclosed herein are synthetically produced as disclosed herein for use in metal recovery, metal mining, metal recycling, metal remediation, pollution control, or any process including metal sequestering. Therefore, the metal-binding proteins and associated methods disclosed herein provide a versatile, easily produced, efficient, and reliable resource for use in any process having a metal-binding aspect.

In some embodiments a metal-binding protein may include any amino acid sequence that may result in a protein that may be useful for chelating or otherwise sequestering metals. Therefore any variation of protein sequence that would functionally result in an equivalent metal binding protein may be useful in embodiments including a metal-binding protein element, by extension.

In some embodiments, MT metal-binding proteins were isolated from brine shrimp of the genus Artemia. Artemia MT are a family of metal binding proteins that are referred to as “isoforms.” Analysis of these proteins' unique amino acid compositions showed each isoform to be essentially equivalent. An isoform has the broadest meaning as understood by a person of ordinary skill in the art, and includes functional analogs of the same protein, or proteins that may have similar physical, biological, chemical, or pharmacological properties. In some embodiments, two isoforms of a protein type may also be isomers. In some embodiments, isoforms may include structural analogs. At least five individual Artemia MT isoforms have been identified as disclosed herein. Unlike MTs from other organisms which share a high degree of sequence homology or similarity, the Artemia metal binding proteins have unexpectedly different structural characteristics but possess a high degree of sequence homology to one another.

Therefore, the present disclosure may provide substantially purified metal-binding proteins for use in removal of metals from metal-containing organic solvents by reversibly binding the metal to the metal-binding proteins immobilized on a solid support. The term “substantially purified”, as used herein, refers to nucleic acids, amino acids or proteins that have been removed from their natural environment, isolated or separated and are at least 60% free, 75% free, to 90% or more free from other components with which they are naturally associated.

In some embodiments, a substantially purified metal-binding protein may have an amino acid sequence of:

(SEQ ID NO: 1)
MDCCKNGCTCAPNCKCAKDCKCCKGCECKSNPECKCEKNCSCNSCGCH

In some embodiments, the metal-binding protein is a recombinant chimeric metal-binding protein for use in removal of metals from metal-containing substrates A recombinant chimeric metal-binding protein has an amino acid sequence of:

(SEQ ID NO: 2)
HHHHHHGSLQDSEVNQEAKPEVKPEVKPETHINLKVSDGSSEIFFKIKKT
TPLRRLMEAFAKRQGKEMDSLTFLYDGIEIQADQTPEDLDMEDNDIIEAH
REQIGGMDCCKNGCTCAPNCKCAKDCKCCKGCECKSNPECKCEKNCSCNS
CGCH

In some embodiments, the metal binding protein comprises amino acids 7-154 of SEQ ID NO:2.

Also within the scope of the present disclosure are metal-binding proteins that are variants of the metal-binding proteins that preserve the protein's metal-binding affinity. In particular, conservative amino acid substitutions within the scope of the present can include any of the following: (1) any substitution of isoleucine for leucine or valine, leucine for isoleucine, and valine for leucine or isoleucine; (2) any substitution of aspartic acid for glutamic acid and of glutamic acid for aspartic acid; (3) any substitution of glutamine for asparagine and of asparagine for glutamine; and (4) any substitution of serine for threonine and of threonine for serine.

A “conservative amino acid substitution” as used herein, refers to alteration of an amino acid sequence by substituting an amino acid having similar structural or chemical properties. Those skilled in the art can determine which amino acid residues may be substituted, inserted, or altered without the metal-binding properties of the proteins disclosed herein.

Other substitutions can also be considered conservative, depending upon the environment of the particular amino acid. For example, glycine and alanine can be interchangeable, as can be alanine and valine. Methionine, which is relatively hydrophobic, can be interchanged frequently with leucine and isoleucine, and sometimes with valine. Lysine and arginine are interchangeable in locations in which the significant feature of the amino acid residue is its charge and the different pKs of these two amino acid residues and where their different sizes are not significant. Still other changes can be considered “conservative” in particular environments, as known in the art.

For example, if an amino acid on the surface of a protein is not involved in a hydrogen bond or salt bridge interaction with another molecule, such as another protein subunit or a ligand bound by the protein, negatively charged amino acids such as glutamic acid and aspartic acid can be substituted with positively charged amino acids such as lysine or arginine and vice versa. Histidine, which is more weakly basic than arginine or lysine, and is partially charged at neutral pH, can sometimes be substituted for these more basic amino acids as well. Additionally, the amides glutamine and asparagine can sometimes be substituted for their carboxylic acid homologues, glutamic acid and aspartic acid.

In one embodiment, an MT protein has the amino acid sequence of SEQ ID NO:1, 2, or 9-20. An MT protein can also comprise conservative variants to the amino acid sequence of SEQ ID NO: 1, 2, or 9-20. In an embodiment, a conservative variant of an MT protein is a conservative variant of an MT protein disclosed herein. In aspects of this embodiment, a conservative variant of an MT protein can be, for example, an amino acid sequence having at least 50%, 55%, 60%, 65%, 70%, 75%, at least 80%, at least 85%, at least 90%, at least 95%, at least 97%, or at least 98%, or at least 99% amino acid sequence identity to SEQ ID NO:1, 2, or 9-20 and which retains metal-binding activity. In other aspects of this embodiment, a conservative variant of an MT protein can be, for example, an amino acid sequence having at most 50%, 55%, 60%, 65%, 70%, 75%, at most 80%, at most 85%, at most 90%, at most 95%, at most 97%, or at most 98%, or at most 99% amino acid sequence identity to SEQ ID NO:1, 2, or 9-20 and which retains metal-binding activity.

In other aspects of this embodiment, a conservative variant of an MT protein can be, for example, an MT protein having 1, 2, 3, 4, 5, 6, 7, 8, 9, 10, 11, 12, 13, 14, 15, 20, 25, 30 or more conservative substitutions, to the amino acid sequence of SEQ ID NO:1, 2, or 9-20. In other aspects of this embodiment, a conservative variant of an MT protein can be, for example, an amino acid sequence having at least 1, at least 2, at least 3, at least 4, at least 5, at least 6, at least 7, at least 8, at least 9, at least 10, at least 11, at least 12, at least 13, at least 14, at least 15, at least 20, or at least 25 conservative substitutions to the amino acid sequence of SEQ ID NO:1, 2, or 9-20 and which retains metal-binding activity. In yet other aspects of this embodiment, a conservative variant of an MT protein can be, for example, an amino acid sequence having at most 1, at most 2, at most 3, at most 4, at most 5, at most 6, at most 7, at most 8, at most 9, at most 10, at most 11, at most 12, at most 13, at most 14, at most 15, at most 20, at most 25, or at most 30 conservative substitutions to the amino acid sequence of SEQ ID NO:1, 2, or 9-20 and which retains metal-binding activity.

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g. by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are MT proteins which have been modified using ordinary molecular biological techniques so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The peptides disclosed herein are not limited to products of any of the specific exemplary processes listed herein.

In addition to substantially full length polypeptides, the present disclosure also provides for biologically active fragments of the full length MT protein as exemplified by SEQ ID NO:1, 2, or 9-20 and which retains metal-binding activity.

As used herein, amino acid sequences which are substantially the same typically share at least 95% amino acid identity. It is recognized, however, that proteins (and DNA or mRNA encoding such proteins) containing less than the above-described level of identity arising as splice variants or that are modified by conservative amino acid substitutions (or substitution of degenerate codons) are contemplated to be within the scope of the present disclosure. As readily recognized by those of skill in the art, various ways have been devised to align sequences for comparison, e.g., Blosum 62 scoring matrix, as described by Henikoff and Henikoff in Proc. Natl. Acad Sci. USA 89:10915 (1992). Algorithms conveniently employed for this purpose are widely available (see, for example, Needleman and Wunsch in J. Mol. Bio. 48:443 (1970).

Therefore, disclosed herein are amino acid sequences 85%, 90%, 95%, 96%, 97%, 98%, or 99% identical to the MT proteins of SEQ ID NO:1, 2, or 9-20 and which retain metal-binding activity.

In other aspects of this embodiment, an MT protein has, e.g., at least one, at least two, at least three, at least four, at least five, at least six, at least seven, at least eight, at least nine or at least ten contiguous amino acid substitutions, deletions, and/or additions relative to SEQ ID NO:1, 2, or 9-20 and which retain metal-binding activity. In yet other aspects of this embodiment, an MT protein has, e.g., at most one, at most two, at most three, at most four, at most five, at most six, at most seven, at most eight, at most nine or at most ten contiguous amino acid substitutions, deletions, and/or additions relative to SEQ ID NO:1, 2, or 9-20 and which retain metal-binding activity.

The disclosed metallothionein metal-binding proteins have been proven to bind to metals, or metal-containing materials, including, but not limited to, aluminum, arsenic, cadmium, chromium, cobalt, copper, europium, gallium, gold, gold nanoparticles, indium, lanthanum, lead, mercury, methyl mercury, nickel, palladium, palladium nanoparticles, platinum, rhodium, ruthenium, selenium, silver, thimerosal (a mercury based preservative), titanium, tributyl tin, uranium, and zinc.

In addition to their metal-binding properties, the metal-binding proteins also exhibit features which render them particularly useful in a wide variety of metal recovery and metal remediation settings. For example, the metal-binding proteins are capable of heavy metal-binding under a range of conditions such as under moderate to high temperature conditions. The proteins are capable of heavy metal-binding at room temperature and therefore particularly ideal for many applications. The metal-binding proteins are also capable of heavy metal-binding within a wide temperature range such as, for example, a temperature range of about 4° C. to about 100° C. Those skilled in the art will appreciate that depending on a particular application or operation in which the metal-binding proteins are to be utilized, a particular temperature range may be preferred for practical or economic reasons. For example, it may be more practical to use the metal-binding proteins “on-site” or at the location of an environmental contamination (which would dictate that particular temperature range that can be obtained within available costs). On the other hand, more effective metal extraction on certain substrates may be achieved by use of the metal-binding proteins under relatively high temperature conditions. Therefore, a suitable range of temperatures for practicing the present methods includes a range of about 4° C. to about 100° C. This range of temperature conditions makes the metal-binding proteins more versatile and useful.

Further, the metal-binding proteins can be utilized as a naked composition, as a metal-binding material, or in association with a support or dispersal means to aid in either the dispersal, handling, packaging or function of the metal-binding protein in metal recovery, metal remediation or metal-binding processes. Such metal-binding proteins are particularly useful in metal recovery, metal remediation and metal-binding processes because they can be more easily and safely used as compared to other methodologies, such as chemical extraction, which exposes the user to toxic or other potentially dangerous types of chemicals. As used herein, the term “metal-binding material” refers to any material comprising a metallothionein protein disclosed herein in any form. A metal binding material can take the form of an aqueous solution of an MT protein, a solid support having associated therewith an MT protein disclosed herein, or any form of an MT protein that can bind metal and remove metal from a substrate.

A variety of solid supports to aid in the handling or dispersal of the metal-binding proteins can be used and include a hydrophilic membrane, partially hydrophilic membrane, composite membrane, porous organic solid support, nonporous organic solid support, porous inorganic solid support, nonporous inorganic solid supports and combinations thereof. If the solid support is a membrane, membranes such as those described in U.S. Pat. Nos. 5,618,433 and 5,547,760, both of which are herein incorporated by reference in their entireties, are exemplary. If the solid support is an inorganic or organic particulate solid support, preferred solid supports include sand, silicas, silicates, silica gel, glass, glass beads, glass fibers, latex beads, magnetic beads, agarose beads, alumina, zirconia, titania, nickel oxide polyacrylate, polystyrene, polyphenol, and others as described in U.S. Pat. Nos. 4,943,375, 4,952,321, 4,959,153, 4,960,882, 5,039,419, 5,071,819, 5,078,978, 5,084,430, 5,173,470, 5,179,213, 5,182,251, 5,190,661, 5,244,856, 5,273,660 and 5,393,892 which are herein incorporated by reference in their entireties, and combinations thereof. Specific examples include flexible membranes, beads or particulates, filters, or any other solid supports known in the art that are useful for separations.

In certain embodiments, the solid support is a resin. Exemplary resins include, but are not limited to, agarose, polystyrene beads, silica, nanoparticles, magnetic beads, fluorinated polymers, polyolefins, polystyrene, substituted polystyrenes, polysulfones, polyesters, polyacrylates, polycarbonates, vinyl polymers, copolymers of butadiene and styrene, fluorinated ethylene-propylene copolymers, ethylenechlorotrifluoroethylene copolymers, nylon and mixtures thereof.

In one illustrative embodiment, the solid support is in the form of a membrane. The membrane may be a polymer, and may be selected from the group consisting of fluorinated polymers, polyolefins, polystyrene, substituted polystyrenes, polysulfones, polyesters, polyacrylates, polycarbonates, vinyl polymers, copolymers of butadiene and styrene, fluorinated ethylene-propylene copolymers, ethylenechlorotrifluoroethylene copolymers, nylon and mixtures thereof.

The metal-binding proteins disclosed herein are associated with a support, such as a resin, by covalent bonding of the metal-binding protein to the resin, or by non-covalent binding such as, but not limited to, electrostatic attractions, dispersion forces, and solvent-mediated forces.

In one embodiment, the metal-binding proteins are associated with a solid support such that a regenerative metal-binding support is provided, wherein the regenerative metal-binding support can bind heavy metals from a substrate, the heavy metals can be released from the regenerative metal-binding support, and the regenerative metal-binding support can be reused to bind heavy metals.

In general, a substrate from which one or more heavy metal species are to be removed is contacted with a metal-binding protein bound to a solid support, where the metal-binding protein has an affinity for the heavy metal. The solid support forms a support for the metal-binding protein and can be in the form of a membrane, beads, or solid support particulates, or any other form commonly used in biochemical or chemical separations. If a membrane or a resin is used as the solid support, the metal-binding protein-solid support composition can be incorporated into a contacting device comprising a housing, e.g., cartridge, containing the disclosed metal-binding proteins by causing solution containing desired ions to flow through the cartridge and thus come in contact with the metal-binding proteins. In one embodiment, the membrane configuration is a pleated membrane, although other membrane configurations, such as flat sheet, stacked disk, or hollow fibers may be used. However, various contact apparatus may be used instead of a cartridge such as, but not limited to, a cassette, syringe, unit, canister, multi-well plate, or filter holder. If a solid support is used, separation columns can be used as are known in the art.

It should be noted, that an additional characteristic feature of the metal-binding proteins are that they are also capable of reversible heavy metal-binding. For example, bound metals can be eluted off or away from the metal-binding proteins using acidic conditions or by instantaneous exchange reactions or inorganic chelators. For example, during incubation of a metal-binding protein with radioactive Cd, the ¹⁰⁹Cd metal exchanges for endogenous metal bound to the metal-binding protein. At about pH 1.0, the metal is released from the protein. Bringing the pH of the solution up to about pH 8.0 regenerates the metal-binding activity of the protein. Therefore, due to the reversible binding characteristics of the metal-binding proteins, also provided herein are compositions, formulations, powders, liquids, devices, or apparatuses comprising the metal-binding proteins which can be utilized more than once.

Another embodiment provides one or more amino acid sequences encoding a recombinant chimeric metal-binding protein. An exemplary amino acid sequence includes SEQ ID NO:2.

The recombinant chimeric metal-binding proteins can have molecular weight of about 17,300 daltons and are able to bind with high affinity to heavy metal ions, including but not limited to, arsenic, zinc, copper, chromium, cadmium, mercury, methyl mercury, cobalt, lead, nickel, platinum, silver, rhodium, palladium, selenium, and gold. The recombinant chimeric metal-binding proteins include therein an amino acid sequence selected from the group consisting of SEQ ID NO:2, sequences greater than 90% identical to the full-length SEQ ID NO:2, and sequences incorporating one or more conservative amino acid substitutions thereof wherein the conservative amino acid substitutions are any of the following: (1) any of isoleucine, leucine and valine for any other of these amino acids; (2) aspartic acid for glutamic acid and vice versa; (3) glutamine for asparagine and vice versa; and (4) serine for threonine and vice versa. Alternative nucleic acid sequences can be determined using the standard genetic code; the alternative codons are readily determinable for each amino acid in this sequence.

One embodiment disclosed herein provides one or more nucleic acid sequences encoding a substantially purified metal binding protein having amino acid sequence analogous to at least one metallothionein protein from an organism including, but not limited to, Artemia, mammals and marine species, or other species having a metallothionein protein with conserved amino acid sequence homology in the cysteine residues, e.g. the metal-binding motifs, as compared to Artemia MT.

Exemplary uses of these for the disclosed metal-binding proteins include pollution control applications such as metal remediation, pollution control, metal recycling or metal mining. For example, the metal-binding proteins can be used to reduce the concentration of heavy metals in an environmental substance. The substance can be a fluid, such as ground water, sludge, waste-water and the like. Additionally, the metal-binding proteins can be incorporated into one or more compositions or devices used for pollution control. For example, the metal-binding proteins can be applied on site in the form of a flocculent or powder, or can be used in treatment plants as part of a membrane filtration or other type of solid support device used for removal of heavy metal from a contaminated substrate.

The metal-binding proteins used in these metal-binding processes can be produced by bioengineering techniques. For example, the metal-binding proteins can be produced by transgenic or modified organisms. Modified organisms include transgenic animals, bacteria, or plants. For example, a modified plant can be a transgenic tobacco plant whose genome has been genetically altered to express one or more metal-binding proteins. A modified organism can also include a plant or biomass that is capable of growing at or within contaminated sites where metal remediation is desired. Extraction of metal contaminants by the modified organisms also concentrates the toxic metals from the contaminated site. This provides the additional advantage of converting the heavy metals to a smaller quantity as well as providing final product that is more easily and safely handled for disposal or further processing.

Methods for reducing the concentration of heavy metals in a substrate include contacting a metal-binding protein with a substrate having heavy metals. In a non-limiting example, a metal-binding protein can be contacted with a substance having a concentration of at least one heavy metal to bind the heavy metal to the metal-binding protein. Subsequently, the bound heavy metal can be separated from the substrate, reducing the concentration of heavy metals in the original substrate.

As mentioned previously, an additional advantageous feature of the metal-binding proteins include their ability to release bound heavy metals using acid extraction, inorganic chelators, and/or exchange reaction technologies. This allows the user, if desired, to elute bound heavy metals off the metal-binding proteins. Once the heavy metals are eluted off the metal-binding proteins, the metal-binding proteins can be regenerated (or recycled) for additional uses in metal extraction. Therefore, also provided herein are methods for reducing the concentration of heavy metals in a substrate using reusable compositions, devices, and apparatuses comprising the metal-binding proteins.

The metal-binding proteins, when used in methods for reducing the concentration of a metal in a substrate can be provided in such a way as is appropriate for the particular use, situation, mode of administration, or environment in which the metal-binding proteins are to be used. For example, when used in metal remediation, or in pollution control, the metal-binding proteins can be coupled to a support, such as a powder and used, for example, as a flocculent to provide a convenient and efficient means of dispersing the metal-binding proteins.

Alternatively, the metal-binding proteins can be provided coupled to a membrane, a semi-permeable membrane, a filter, a resin, or any other means appropriate for allowing sufficient exposure of the metal-binding proteins to the heavy metal containing substrate so as to bind or sequester the heavy metals from the substrate. A membrane or filter comprising the metal-binding proteins provides a particularly efficient means of treating ground water or waste water, as contaminated water can be purified by passage through the membrane or filter without further clean up as is required in chemical extraction processes. Coupling the metal-binding proteins to a support or supporting matrix also affords easier handling of the metal-binding proteins especially when used in large scale or industrial applications.

Use of the metal-binding proteins are not limited only to those methods where removal of heavy metals is desired, but can also include methods where recovery or concentration of heavy metals in a substance is to be achieved. For example, the metal-binding proteins can be used for metal mining or recovery, such as in the recovery of precious or rare metals including, but not limited to, gold, platinum and silver, or can be used to concentrate metals in hazardous conditions, such as hazardous waste containing radioactive metals. Such hazardous metal waste can result either from numerous research, commercial, or industrial uses.

Use of the metal-binding proteins in concentrating radioactive metals from waste also reduces the amount or quantity of hazardous waste to be disposed. Reducing the quantity of hazardous metal waste also reduces the level of radioactivity to which certain individuals are exposed.

In some embodiments, methods for heavy metal extraction may include liquid-liquid extraction involving dissolving a metal-binding protein in an aqueous solution. The aqueous solution can then be intimately mixed with a metal-containing solution (such as an organic solution) which is substantially immiscible in the aqueous solution. Upon mixing of the two immiscible layers the organic layer may have a significantly lower amount of heavy metal and the aqueous layer will have substantially extracted the heavy metal from the metal-containing solution. In some embodiments, the dissociation constant of the metal-binding protein may be related to the pH of the aqueous solvent in which it is dissolved.

In some embodiments, heavy metal extraction or recovery may include the extraction of a metal from an organic solvent by using an aqueous solution that includes a dissolved metal-binding protein.

In some embodiments, the method disclosed herein includes an aqueous solution comprising a metal bound to a metal-binding protein having an amino acid sequence of SEQ ID NO:1, 2, or 9-20, wherein the aqueous solution is contacted with a metal containing organic solution, which separates the metal from the organic solution and brings the metal into the aqueous solution.

A metal-binding protein can be prepared in an aqueous solution at a concentration of about 1 mg/ml to about 100 mg/ml, about 1 mg/ml to about 50 mg/ml, about 3 mg/ml to about 20 mg/ml, about 3 mg/ml to about 40 mg/ml, about 5 mg/ml to about 15 mg/ml, about 6 mg/ml to about 10 mg/ml, or any concentration bounded by or between these values.

In some embodiments, the aqueous solvent may be primarily water, or an aqueous buffer solution. The aqueous solvent may comprise other miscible solvents that may result in a useful solution for maximum heavy metal extraction capability. The aqueous solvent may comprise other miscible solvents that may result in a useful solution for dissolving metal-binding proteins.

In some embodiments, the organic solvent may include any organic solvent that may be immiscible in water. Some embodiments may include a mixture of organic solvents. In some embodiments, any carbon-based solvent may be an organic solvent such as a hydrocarbon, a halocarbon, a halohydrocarbon, or an ether. Some organic solvents may include any nonpolar solvent. Organic solvents may include any nonpolar solvent that may be useful for dissolving heavy metals. Some organic solvents may include but are not limited to: halocarbons, such as, dichloromethane, chloroform, fluorocarbons, etc.; diethyl ether; hexane; benzene; toluene; xylene; etc.

In some embodiments, the volume ratio of aqueous solution to organic solution may be from about 0.1 to 10 (aqueous solution/organic), about 0.5 to 5, about 0.8 to 1.2, or about 1 to 1, or any ratio bounded by or between any of these values.

Mixing can be performed by inversion, sonication, stirring, shaking, or any other useful method or combinations of methods that would result in a useful contacting of the metal binding protein in the aqueous phase, with the metal in the organic phase. The mixing process may be performed for any useful time period, such as but not limited to, about 1 min to about 5 min, about 5 min to about 1 hr, about 1 hr to about 6 hr or for any time frame bounded by or between any of these ranges. In some embodiments, the metal is chelated or otherwise held or substantially sequestered by the metal binding protein.

After mixing, the solution may be allowed to separate for about 1 min to about 6 hr, from about 1 min to about 10 min, from about 10 min to about 30 min, from about 30 min to about 1 hr, from about 1 hr to about 3 hr, or any useful time frame bounded by or between any of these values that would result in a substantially separate organic and aqueous phase.

In some embodiments, at least 90%, 95%, 99% of the mass of the aqueous liquid is not dispersed in the organic liquid. In some embodiments, at least 70%, 80%, 90%, 95%, or 99% of the metal has been transferred from the organic phase to the metal-binding protein-containing aqueous phase.

In some embodiments, after the aqueous and organic phases are allowed to separate into two substantially separate phases, the aqueous layer may be removed. Devices for extraction may include any device that may facilitate the extraction of the aqueous phase from the organic phase or vice versa.

In some embodiments, multiple mixing, separating, and/or removing steps may be performed.

In some embodiments, the pH may be reduced to release the bound metal from the protein. For example, the metal-binding protein may release the bound metal at a pH of about 0.5, about 1, about 1.5, about 2, or about 2.5, or any pH bounded by or between any of these values. At low pH, the metal may then be filtered from the aqueous solution. In some embodiments, at least about 70%, 80%, 90%, 95%, or 99% of the metal is removed from the aqueous solution.

In some embodiments, after the pH has been reduced and the metal released from the protein, the pH may be increased to regenerate the metal-binding affinity of the protein. For example, the pH at which the metal-binding protein regenerates its metal-binding affinity after metal release may be at a pH of about 5, about 5.5, about 6, about 6.5, about 7, about 7.5, about 8, about 8.5, or about 9, or any pH bounded by or between any of these values.

In some embodiments, a certain pH may cause an increased binding affinity for one metal over another metal.

Some embodiments may include methods for reducing the concentration of a metal in an organic solvent comprising the steps of: intimately mixing a metal-binding protein containing aqueous solution, as disclosed herein, with the metal-containing organic solvent to bind the metal to the metal-binding protein; and separating the aqueous solution containing the metal-binding protein and bound metal from the organic solvent.

In some embodiments, the organic solvent that includes the metal may be a fluid, a liquid, a gel, a suspension, an emulsion, a metallic colloid, etc.

In some embodiments, the metal-binding protein may be substantially purified before reconstitution in the aqueous solvent.

In some embodiments, the aqueous metal-binding protein solution may have a higher affinity for a metal than the organic solution.

Metal-binding proteins disclosed herein, when used in methods for reducing the concentration of a metal in a organic solvent can be provided in such a way as is appropriate for the particular use, situation, mode of administration or environment in which the metal-binding proteins are to be used. For example, when used in metal remediation, or in pollution control, the metal binding proteins can be coupled to a support, such as a powder and used, for example, as a flocculent to provide a convenient and efficient means of dispersing the metal binding proteins. Use of the metal-binding proteins in concentrating radioactive metals from waste also reduces the amount or quantity of hazardous waste to be disposed of. Reducing the quantity of hazardous metal waste also reduces the level of radioactivity to which certain individuals are exposed.

Methods for reducing the concentration of heavy metals in a substance include producing the metal-binding proteins in a modified organism. Modified organisms include, for example, transgenic organisms or transgenic hosts. For example, hosts or organisms such as shrimp, plants, bacteria, yeast, or algae can be modified using molecular and genetic engineering techniques well known in the art. Using these techniques, which are described for example, in Sambrook et al., Molecular Cloning: A Laboratory Manual (New York: Cold Spring Harbor Press, 2001); Ausubel et al. Current Protocols in Molecular Biology (Wiley Interscience Publishers, 1995); US Dept Commerce/NOAA/NMFS/NWFSC Molecular Biology Protocols (URL:http://research.nwfsc.noaa.gov/protocols.html); or Protocols Online (URL:www.protocol-online.net/molbio/index.htm), organisms whose genomes are modified so as to result in expression of a metal-binding protein are provided. Modified organisms can be made and used to produce these metal-binding proteins, and the metal-binding proteins useful in the methods provided herein.

A modified organism producing a metal-binding protein includes a modified organism producing at least one metal-binding protein having an amino acid sequence at least 85%, at least 90%, or at least 95% identical to SEQ ID NO:1, 2, or 9-20.

Alternatively, production or expression of the metal-binding proteins from modified organisms is not limited to genomic expression of the metal-binding proteins, but also includes epigenetic expression of the metal-binding proteins from the modified organisms. Methods and techniques for obtaining epigenetic expression from a modified organism include, for example, adenoviral, adeno-associated viral, plasmid and transient expression techniques which are known in the art.

Also provided are methods for producing the metal-binding proteins. For example, a method for producing a metal-binding protein having an amino acid sequence at least 85%, at least 90%, or at least 95% identical to SEQ ID NO:1, 2, or 9-20 includes providing an expression system, producing a metal-binding protein using the expression system and purifying or isolating the metal-binding proteins to obtain a metal-binding protein.

Expression systems can be systems such as traditional manufacturing plants. Biomanufacturing systems using genetically engineered organisms (produced as described herein) capable of producing the r metal-binding proteins can be used to produce the metal-binding proteins. For example, bacteria containing a metal-binding protein expression vector can be cultured on large or small scale (depending on the particular need). The metal-binding proteins can then be purified from the bacterial broth and used in metal-binding processes.

Therefore, a metal-binding protein can be produced by expression of a nucleic acid sequence encoding a metal-binding protein in a modified organism or host cell.

The expressed metal-binding proteins are purified using standard techniques. Techniques for purification of cloned proteins are well known in the art and need not be detailed further here. One particularly suitable method of purification is affinity chromatography employing an immobilized antibody to a metal-binding protein. Other protein purification methods include chromatography on ion-exchange resins, gel electrophoresis, isoelectric focusing, and gel filtration, among others. Alternatively, the metal-binding proteins can be purified following their expression from modified organisms by methods such as precipitation with reagents (e.g. ammonium sulfate, acetone or protamine sulfate as well as other methods known in the art).

Also disclosed herein are systems, devices and methods for removing metals, such as mercury-containing thimerosal, from medications using the disclosed metallothionein proteins are provided. In one embodiment, the thimerosal is removed prior to the dosing procedure from a bulk biological material.

The dosing devices, systems and methods disclosed herein remove substantially all of the thimerosal from the medication. In one embodiment, greater than about 90% of the thimerosal is removed. In another embodiment, greater than about 91%, 92%, 93%, 94%, 95%, 96%, 97%, 98% or 99% of the thimerosal is removed. In another embodiment, the present methods produce medications having less than about 1 μg of thimerosal per dose of medication. In another embodiment, each dose of medication contains less than about 0.7 μg thimerosal. In another embodiment, each dose of medication contains less than about 0.5 μg of thimerosal. In yet another embodiment, each dose of medication contains less than about 0.1 μg of thimerosal.

As used herein, the term “medication” refers to any pharmaceutical preparation to be administered to a subject and which contains a heavy metal or heavy metal-containing compound including, but not limited to, thimerosal. The term medication includes, but is not limited to, injectable medications such as, but not limited to, vaccines, immunogenic compositions, gene therapy agents and any type of injectable agent, liquid pharmaceutical compositions, colloidal pharmaceutical compositions, suspension pharmaceutical compositions, aerosols and dry powders. For the purposes of this disclosure, medication and bioactive material are used interchangeably.

As used herein, medication is defined as any material suitable for administration to a mammal through any route. Non-limiting examples of medications include vaccines, plasma-derived products such as immune globulin and anti-toxins or anti-venoms and drugs including chemicals and biologicals including, but not limited to, proteins, peptides, hormones, polysaccharides, etc. Medications can also refer to immunogenic compositions, liquid pharmaceutical compositions, colloidal pharmaceutical compositions, suspension pharmaceutical compositions, aerosols and dry powders. Medication and bioactive material may be used interchangeably and are considered equivalent terms for the purposes of this disclosure. Routes of administration addressed by the devices and methods disclosed herein include, but are not limited to, intravenous, subcutaneous, intradermal, and intramuscular injection; intravenous infusion; oral; inhalation; intraocular and other routes of administration known by medical professionals.

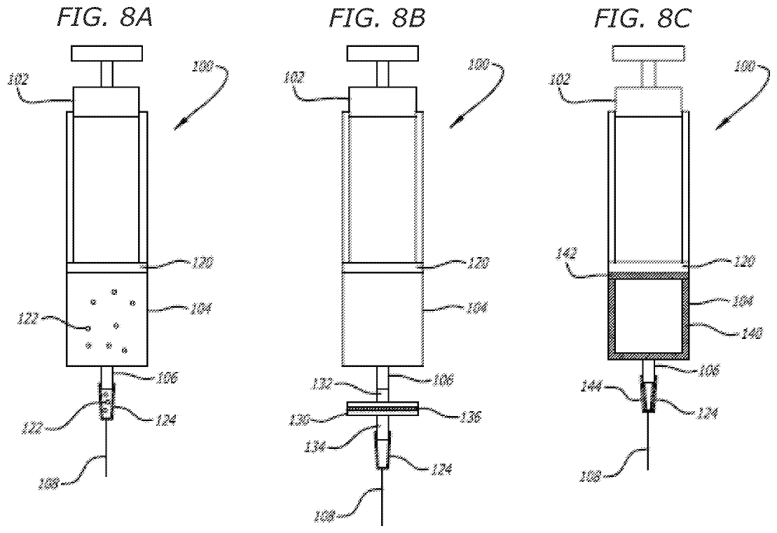

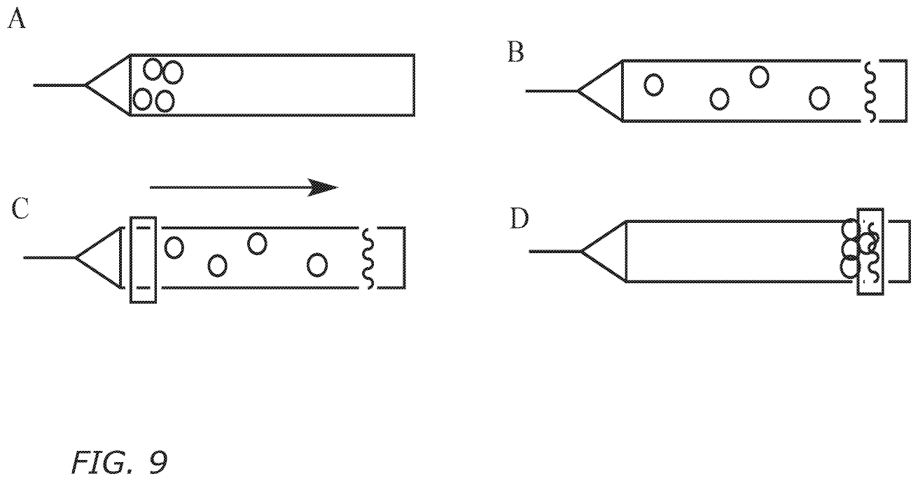

In one embodiment, a dosing device has MT associated therewith to remove thimerosal from a thimerosal-containing solution passing through the dosing device. One exemplary dosing device depicted in FIG. 8 is a syringe. The MT can be associated with the dosing device in several ways. FIG. 8 depicts a syringe 100 generally having a plunger 102, a barrel 104 and a luer tip 106 for attachment to a needle 108. Needle 108 typically has a complimentary luer hub 124 for attaching to luer tip 106. Syringes can be manufactured to a variety of specifications and can have more or less components than depicted in FIG. 8. Optionally a gasket 120 is present to provide a seal between plunger 102 and barrel 104. In FIG. 8A, MT is bound to a solid support in the form of a bead 122, and a plurality of beads 122 having at least one MT protein bound thereto are disposed within barrel 104 of syringe 100 and/or luer hub 124 of needle 108 prior to a thimerosal-containing solution entering syringe 100. Injectable materials drawn into syringe 100 contact MT-coated beads 122 and thimerosal in the injectable material becomes bound to the MT protein. The beads can be of any size or shape compatible with their use. Beads useful within the luer hub 124 of needle 108 may be of a different size than those used within a reservoir or syringe barrel 104. Furthermore, beads within the barrel 104 of syringe 100 or luer hub 124 of needle 108 are retained within the barrel 104 or luer hub 124 after passage of the injectable material and do not pass into the subject or patient. The beads are retained by means including, but not limited to, presence of a membrane or mesh in barrel 104 or luer hub 124 with pore sizes smaller than the beads and/or the size of the beads exceeding the size of any exit ports of the barrel 104 or luer hub 124 such that the beads do not leave the barrel 104 or luer hub 124 with the injection material. A method for using the device of FIG. 8 is depicted in FIG. 9.

Beads suitable for coating with chimeric MT proteins include, but are not limited to, biocompatible polymers such as fluorinated polymers, polyolefins, polystyrene, substituted polystyrenes, polysulfones, polyesters, polyacrylates, polycarbonates; vinyl polymers, copolymers of butadiene and styrene, fluorinated ethylene-propylene copolymers, ethylenechlorotrifluoroethylene copolymers, nylon, and mixtures thereof.

### EXAMPLES

In accordance to the teachings of the present disclosure, the following exemplary protocols illustrate methods useful in the production, purification and analysis of metal-binding proteins.

### Example 1

### Cloning of Artemia Metal-Binding Protein

The following techniques were utilized to provide nucleic acid sequence encoding a Artemia metal-binding protein. First, metal-binding proteins (metallothionein, MT) from brine shrimp (Artemia) were isolated and purified. N-terminal amino acid sequence analysis was performed on the isolated metal-binding protein. Amino acid sequence analysis indicated that the metal-binding motif of the first six cysteine residues of the Artemia metal-binding protein was conserved when compared to rabbit and human MTs, indicating the importance of these amino acid residues in the protein's metal-binding function.

Using this N-terminal amino acid sequence information, oligonucleotide primers corresponding to the N-terminal amino acid sequence were constructed as known in the art. These oligonucleotide primers were used to amplify, by polymerase chain reaction (PCR) potential candidates for a MT gene sequence encoding at least one of the target metal-binding proteins from brine shrimp (Artemia). The PCR product was purified using QIAPREP® spin columns (Qiagen, Inc.) and cloned into the TA cloning vector CR2.1 (Invitrogen) using the manufacturers protocol. Electrocompetent Escherichia coli (Sure Shot cells from Invitrogen) were transformed with the recombinant vector and plated onto LB agar plates containing ampicillin (100 μg/mL) and 1% glucose. The plates were placed at 37° C. overnight. Individual colonies were picked and used to inoculate 5 mL of LB broth supplemented with ampicillin and 1% glucose. The cultures were incubated overnight in a rotary incubator at 37° C. Plasmid was isolate from 2 mL of the cell suspension using QIAPREP® spin columns. The plasmid was then sequenced on a LICOR® 4200L using the M13 universal forward and reverse primers. Once verified and determined to be a sequence encoding a metal-binding protein, the brine shrimp MT gene was subcloned into the bacterial expression vector pTMZ. Based upon the identified MT encoding sequence, the amino acid sequence of the first novel metal-binding proteins was determined.

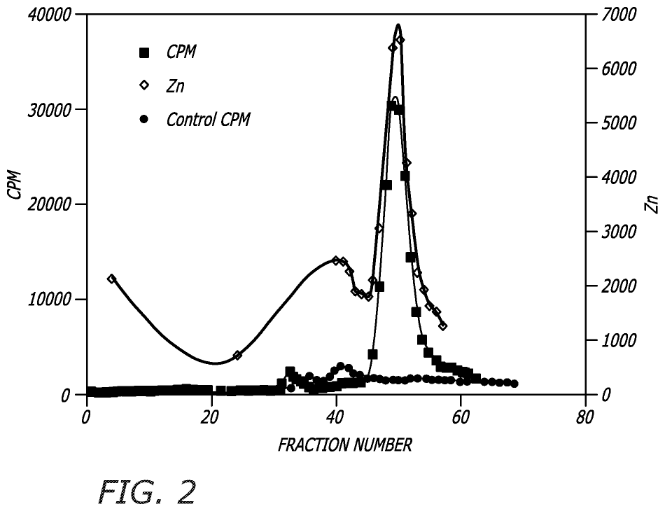

FIG. 2 details an exemplary elution profile utilizing an exemplary metal-binding protein. This profile was obtained utilizing the following exemplary protocol. E. coli (Strain ER 2566) were transformed with a plasmid expression vector containing the MT gene sequence of SEQ ID NO. 1 in pTMZ. Bacteria were grown in LB broth containing 1% glucose at 37° C. to an A₆₀₀ of 0.60. The bacterial cells were collected and resuspended in LB broth containing 0.1% glucose and incubated for 45 min at the same temperature. Isopropyl b-D-thiogalactopyranoside (IPTG) was added to a final concentration of 0.1 mM. The bacterial cells were incubated for about 16 hr. Non-transformed bacteria were used as controls. The cells were collected by centrifugation and sonicated in 10 mM Tris, pH 8.0, 5 mM dithiothreitol (DTT) and 0.5 mM phenylmethylsulfonylfluoride (PMSF). The homogenate was centrifuged at 150,000×g for 1 hr at 4° C. The supernatant was collected and incubated with 2 μCi of ¹⁰⁹Cd at room temperature. The radiolabeled supernatant was then applied to a G-50 molecular exclusion column and eluted with 50 mM Tris, pH 8.0. Five milliliter fractions were collected and assayed for radioactivity (CPM) and zinc (Zn), the zinc being an endogenous metal that associates with the exogenous metal-binding protein expressed by the transformed bacteria. Each fraction eluting from the column was assayed for Zn by ICPMS (Inductively Coupled Plasma Mass Spectroscopy).

The cloned Artemia MT protein has the amino acid sequence of SEQ ID NO:1:

| | |
|---|---|
| MET ASP CYS CYS LYS ASN GLY CYS THR CYS ALA PRO ASN CYS LYS | 15 |
| CYS ALA LYS ASP CYS LYS CYS CYS LYS GLY CYS GLU CYS LYS SER | 30 |
| ASN PRO GLU CYS LYS CYS GLU LYS ASN CYS SER CYS ASN SER CYS | 45 |
| GLY CYS HIS | 48 |

### Example 2

### Isolation and Characterization of Metallothionein Proteins

As a preliminary step in the isolation of the metal-binding proteins, Artemia brine shrimp were grown in artificial seawater (AS) (422.7 mM NaCl, 7.24 mM KCL, 22.58 mM MgCl₂.6H₂O, 25.52 mM MgSO₄.7H₂O, 1.33 mM CaCl₂.2H₂O, and 0.476 mM NaHCO₃). Artemia cysts (2.5 g) were incubated for 48 hr in 250 mL of AS supplemented with antibiotics at 30° C. and rotation at 125 rpm. After 24 hr, phototropic Artemia were collected, cultured for an additional 24 hr and then collected by cloth filtration. The shrimp were weighed and, if not used immediately, stored at −80° C.

The Artemia were then homogenized in homogenization buffer (HB) (10 mM Tris-HCl (pH 8.0), 0.1 mM DTT, 0.5 mM PMSF and 10 μg/mL soybean trypsin inhibitor) and resuspended in HB at 4 mL/gm wet weight of shrimp. The homogenate was passed through a Yamato LH-21 homogenizer three times at a setting of 800 rpm, filtered through CALBIOCHEM® MIRACLOTH™ (EMD Millipore) and the filtrate centrifuged in a SORVALL® SA-600 rotor (Thermo-Fisher) at 14,300 rpm, 4° C. for 30 min. The lipid layer on top of the supernatant was removed by vacuum aspiration and the lower supernatant layer collected and centrifuged in a Beckman 50.2TI rotor at 40K rpm, 4° C. for 90 min. Again, the upper lipid layer was removed and the lower supernatant recentrifuged at 150K (150K sup). The 150K sup was then used immediately or stored at −80° C. If used immediately, this product was then subjected to gel filtration as follows. The gel filtration studies verified the metal-binding proteins' ability to bind to heavy metals.

### Gel Filtration Studies

The 150K sup was centrifuged in a SORVALL® SA-600 rotor at 8,500 rpm and 4° C. for 30 min. The resulting supernatant was then filtered through a HPLC certified 0.45 micron LC13 ACRODISC® filter (Pall Corp.). A 20 mL aliquot of filtered 150K supernatant was incubated at 4° C. for 20 min with 2 μL of ¹⁰⁹Cd (0.066 μCi) to radiolabel the metal-binding proteins. The sample was then applied to a SEPHADEX® G-50 molecular weight exclusion column (2.6 cm×94 cm) previously equilibrated with 50 mM Tris-HCl (pH 8.0) saturated with N₂. One molar DTT (2 μL) was added to fractions 60-100 prior to sample loading in order to maintain reducing conditions in the fractions containing the low molecular weight metal-binding proteins. The column was eluted with 50 mM Tris (pH 8.0) at a flow rate of 20 mL/hr while monitoring the eluate at 280 nm. During the elution period, the buffer reservoir was continually purged with N₂. Samples used for amino acid analysis were not radiolabeled.

The ¹⁰⁹Cd content (CPM) of the column fractions was determined with an AUTO-LOGIC™ gamma counter (ABBOTT Laboratories). Zinc content was measured by Flame or Furnace Atomic Absorption Spectroscopy and expressed as PPB zinc/fraction. Prior studies indicated that two classes of metal-binding proteins were present, one class being a high molecular weight fraction. However, the majority of ¹⁰⁹Cd eluted with a low molecular weight class of zinc-containing metal-binding protein. As shown in FIG. 1, radioactive metal-binding protein had a elution peak corresponding to that for Zinc (roughly, fraction #50). The protein concentration of the SEPHADEX® G-50 fractions was determined with a PIERCE™ BCA Total protein assay kit (Thermo-Fisher) according to manufacturer's protocol. The distinct structural features of the metal-binding proteins of the present disclosure were then identified in the following studies.

### Metal-Binding Protein Characterization Studies

Chromatographic and molecular weight studies were performed to ascertain structural features of the metal-binding proteins. Using anion exchange and reverse phase chromatography techniques metal-binding proteins from Artemia were purified and determined to have molecular weights and amino acid sequence length unexpectedly lower than other known metal-binding proteins. Under SDS-PAGE conditions, Artemia metal-binding proteins have molecular weight of about 5.8 kDa as compared to 6-7 kDa for metal-binding proteins from other mammalian species. Protein analysis of Artemia metal-binding proteins indicates a sequence length of 48 amino acids. The Artemia MT amino acid sequence was unexpectedly and significantly shorter in length than other known metal-binding proteins, which range in length from 60 to 68 amino acid residues.

### Example 3

### Cloning and Sequencing of a Gene Encoding Artemia Metal-Binding Protein

Total RNA was isolated from 48 hour nauplii (the larval stage of Artemia) using the RNAZOL® method. Forty-eight hour nauplii samples were prepared as described above in Example 1. The POLYTRACT® Procedure (Promega) was then used to isolate mRNA from the total RNA samples. cDNA was generated from the mRNA using SUPERSCRIPT® and 3′ RACE Kit procedures (Thermo-Fisher) and then subjected to the synthesis reaction (500 ng Artemia mRNA (25 μL), DEPC H₂O (30 μL), 10 μM AP (5 μL). This mixture was incubated for 10 min at 70° C., then placed on ice for 1-2 min. Volatilized liquid was collected by centrifugation for 10 sec at 10,000 rpm.

A cDNA solution was then prepared from the above RNA cocktail by adding 10×PCR buffer, 25 mM MgCl₂, 10 mM dNTP, and 0.1 mM DTT and the resulting cDNA solution was then mixed and incubated at 42° C. for 5 min. Five (5) μL of SUPERSCRIPT II® RT was added and the mixture incubated at 42° C. for 50 min for cDNA synthesis. The reverse transcription reaction was terminated by incubating the solution for 15 min at 70° C., 5 μL of RNase was then added and the solution incubated for 20 min at 37° C. The final solution containing Artemia cDNA was then stored at −20° C. until used for PCR amplification as described below.

The initial PCR Primer Sequences used were as follows: the 5′ primer (N-terminal side) was designated “MT-Not I” (SEQ ID NO:3) and the 3′ primer (C-terminal side) was designated “dT-Spe I” (SEQ ID NO:4, SEQ ID NO:5, SEQ ID NO:6)

SEQ ID NO: 3
5′-ACC TAT GCG GCC GCA AAT GGA CTG CTG CAA GAA C-
3′
SEQ ID NO: 4
5′-GCA CCA ACT AGT GCC TTT TTT TTT TTT TTT A-3′
SEQ ID NO: 5
5′-GCA CCA ACT AGT GCC TTT TTT TTT TTT TTT C-3′
SEQ ID NO: 6
5′-GCA CCA ACT AGT GCC TTT TTT TTT TTT TTT G-3′.

The above 5′ and 3′ primers were then used in an amplification cocktail (10×PCR Buffer, 25 mM MgCl₂, 10 mM dNTP, 10 μM dT-Spel, and 10 μM MT-Not I) and a Gem 50 wax bead was added to the tube and the tube incubated at 80° C. for 2-3 min. Upon hardening of the wax at room temperature for 10-15 min, the Artemia cDNA mixture and Taq polymerase were layered on top of the hardened wax. This final mixture was then subjected to PCR amplification of an initial denaturation for 3 min at 95° C., followed by 29 cycles of 94° C. for 1 min, 49° C. for 1 min, 72° C. for 1 min and then hold for 72° C. for 10 min and then stored at 4° C.

Once amplified, the PCR product was verified for successful amplification on a 1.2% agarose gel. The PCR product was then purified for subsequent cloning using QIAQUICK® Gel Extraction (Qiagen). The following primers which contain modifying restriction sites incorporated into their sequence were used to amplify and subclone the purified PCR product containing brine shrimp Artemia metal-binding protein gene sequences.

MT Nco I (5′ primer containing an Nde I site):

SEQ ID NO: 7
5′-GCT ACA CAT ATG TCC ATG GAC TGC TGC AAG AAC-3′

MT Sal I (3′ primer containing Sal I site):

SEQ ID NO: 8
5′-ACG AAC GTC GAC GCC TTT TTT TTT TTT TTT A-3′

Using the MT Nco I and MT Sal I primers, with an annealing temperature of 72° C. for 1 min, the Artemia MT nucleotide sequence was amplified and then subsequently subcloned into the pGEM3 vector's Eco RI site. Once subcloned, the cloned metal-binding protein gene can then be easily modified or further processed for use in expression, production or other methods requiring use of an isolated nucleic acid encoding a metal-binding protein.

The entire coding sequence for MT gene was then determined using a LICOR® 4200L DNA sequencer. Sequence comparison studies of the MT gene from Artemia indicate it to have unexpectedly different sequence as compared to other known metal-binding protein genes. When the Artemia MT gene sequence was aligned with that of equine and human MT, homology was observed at the locations of the metal-binding cysteine residues. The ability of the exemplary metal-binding protein of the present disclosure to bind heavy metals was then confirmed in the following studies.

### Example 4

### Transgenic Tobacco Expression of Artemia MT

The following provides an exemplary study which can be performed on any of the novel metal-binding proteins to aid in the verification of a protein as a metal-binding protein. For example, the metal-binding proteins are capable of binding heavy metals such as zinc, cadmium and copper. The ability of an isolated protein to bind heavy metals was described and detailed in the disclosed transformation of E. coli with an exemplary MT and shown, as indicated in FIG. 1.

As described previously, modified organisms useful for producing the novel metal-binding proteins can be made following the teachings provided herein. An exemplary modified organism includes a transgenic tobacco plant which is particularly useful in the methods described herein.

The cDNA for MT cloned into TOPO.CR2 vector is referred to as pARTₘₜ. The coding sequence for the MT was cloned into a pUC18 based plasmid containing the omega 5′ untranslated region of the TMV coat protein in frame with the multiple cloning site. This was accomplished by amplification of the MT coding sequence from pARTₘₜ₁ using PCR primers containing an Nco I restriction site on the 5′ primer and a Sal I site on the 3′ primer. The PCR product and vector were each restricted with Nco I and Sal I and purified. The PCR product was then ligated into the vector using T4 DNA ligase. The ligation mixture was used to transform DH5a cells by electroporation. LB media was inoculated with individual colonies and grown overnight. Plasmid was isolated and sequenced to verify the presence and integrity of the MT coding sequence.

The Eco RI/Xba I cassette was removed and cloned into the corresponding sites on the plant expression vector pSS. The pSS vector contains the constitutive CMV promotor and transcription terminator sequence in frame with the multiple cloning site. The resultant pSSₘₜ construct was propagated in DH5a cells, isolated and sequenced to verify the presence and integrity of the MT gene as described above.

### MT Expression in Tobacco Leaves

A. tumefaciens were transformed with the cytosolic pSSₘₜ construct by electroporation and grown overnight at 27° C. in YEB medium, pH 7.4, containing antibiotics. The cells were collected and resuspended in induction medium (YEB, pH 5.8, antibiotics and 20 μM acetosyringone) and grown overnight at 27° C. The next morning the cells were collected by centrifugation and resuspended in infiltration medium (MMA buffer containing antibiotics and 200 μM acetosyringone) to an A₆₀₀ of 1.5 and incubated at room temperature for 2 hr. Tobacco (Nicotiana tabacum) leaves were submerged in the bacterial suspension and placed in a vacuum dessicator. The leaves were infiltrated under a vacuum of 30-40 mbar. The leaves were placed at room temperature for 72 hr, then ground to a fine powder in liquid nitrogen and extracted with 10 mM Tris pH 8.0, 0.05 mM DTT, 1 mM PMSF. The solution was clarified by centrifugation at 30,000×g and the supernatant assayed for MT using a ¹⁰⁹Cd metal-binding assay. Metal-binding activity is evident in the leaves containing the gene for Artemia MT (Table 1).

TABLE 1

| Treatment | Bound Cd (CPM) |
|---|---|
| Buffer | 747 |
| Untreated Leaves | 5052 |
| Infiltrated Leaves I | 12874 |
| Infiltrated Leaves II | 12763 |

### Stable Transformation of Tobacco

A suspension of A. tumefaciens transformed with pSSₘₜ were grown as described above. Tobacco leaves were cut into small pieces (without the central vein) and transferred into sterile weck glasses containing 50-100 mL of bacterial suspension (A₆₀₀ about 1.0) and incubated at room temperature for 30 min. The leaf pieces were then transferred onto sterile WHATMAN® 3MM filter paper pre-wetted with sterile water in plastic petri dishes. The dishes were sealed with saran wrap and incubated at 26-28° C. in the dark for two days. The leaf pieces were then washed with sterile water containing antibiotics and transferred onto MS II agar plates. The pieces were incubated at 25° C. for 3-4 weeks with a 16 hr photoperiod. When shoots began to form, the shoots were removed and transferred onto MS III agar plates and incubated at 25° C. with a 16 hr photoperiod until roots began to form. The small plants were transferred into Weck glasses containing MS III medium and incubated at 25° C. with a 16 hr photoperiod for about two weeks. The young plants were then planted into soil. Young leaves from the plants were collected and assayed for MT activity as described above to determine the transgenic plants.

### Example 5

### Polymer Membranes for Toxic Metal Removal from Water

Metallothionein was extracted from Artemia embryos as described above. The protein extract (80 mL) was placed in a boiling water bath for 15 min. The solution was centrifuged at 30,000×g (16,000 rpm in a SA600 rotor) for 30 min at 4° C. The supernatant containing the MT was transferred to a clean tube containing 60 μL of ¹⁰⁹Cd (Amersham Biosciences). The solution was mixed well and allowed to stand at room temperature for 5 min. This allows for exchange of the radioactive cadmium onto the MT and provides us with a method for detecting the protein during its purification. The solution was then applied to a 100×4.8 cm G-50 molecular exclusion column and eluted with nitrogen saturated 50 mM Tris, pH 8.0. Fifteen milliliter fractions were collected into tubes containing 25 μL of 1M DTT. The peak metal-binding activity were pooled and stored at 4° C. The solution is referred to as MT.

### Metal-Binding at Neutral pH

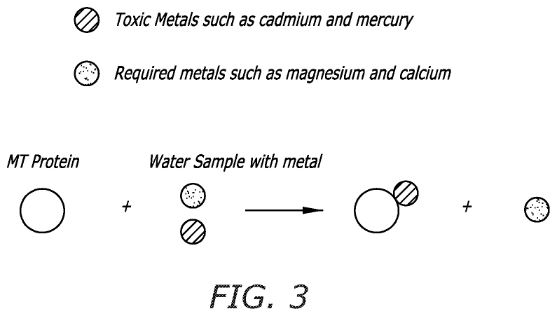

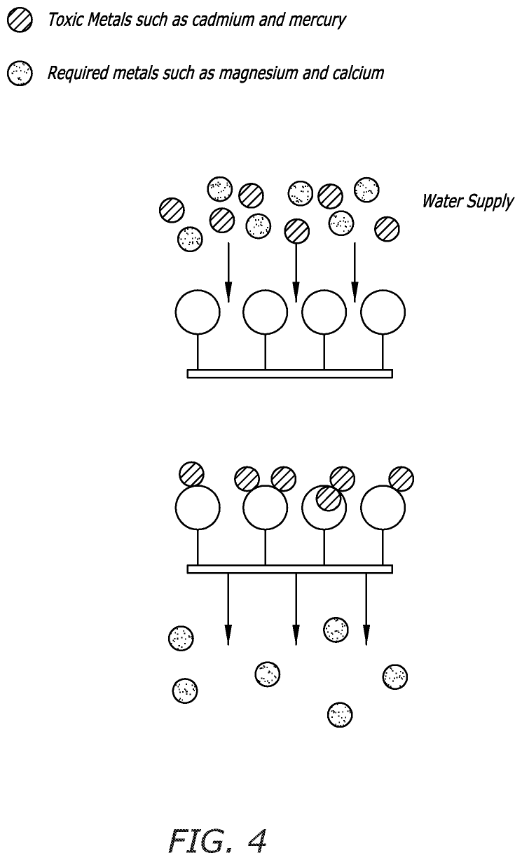

PALL BIODYNE® membranes (Biodyne A and Biodyne B, 0.45 μm, Lot numbers 002245 and 035241, respectively) were used as a solid support for these experiments. A 1 cm² piece of membrane was placed in a 10 mL MILLIPORE® glass frit filtering unit. Ten milliliters of MT was passed through the membrane under vacuum at a flow rate of approximately 100 mL/min (See FIGS. 3 and 4 for a schematic of the procedure). The flow through was collected for protein analysis. Next, 10 mL of a solution of cadmium (0.1 μg/mL of CdCl₂ and 10 μL ¹⁰⁹Cd in 50 mL of water) was passed through the membrane under vacuum. The membrane was then washed twice, each with 10 mL of PBS. Five milliliters of the pooled eluate was analyzed of radioactivity. The membrane was removed from the filtering unit, place in a 12×75 mm centrifuge tube and analyzed for radioactivity in an LKB gamma counter. As a control, the procedure was repeated with a second membrane that had not been treated with MT. This membrane is referred to as the “blank.” The results are shown below in Table 2.

TABLE 2

| Sample | MT Membrane | Blank |
|---|---|---|
| Biodyne A | 152,876 | 3768 |
| Biodyne B | 158,762 | 1774 |

The results demonstrate that membrane-bound MT is capable of removing cadmium (as ¹⁰⁹Cd) from a solution of the metal passed through the membrane. Membranes without MT remove little, if any, metal from the solution.

### Metal-Binding at Varying pH

The next series of experiments were to determine the effect of extremes of pH on the metal-binding activity of the protein on the membrane. A fresh sample of MT was prepared for these studies. The solution of cadmium used for these experiments was prepared as follows: 2 μL of ¹⁰⁹Cd was added to 1 mL of an aqueous solution of CdCl₂ (1 ppm). Then 100 μL of this radioactive cadmium solution was added to 10 mL of each of the following solution: PBS, 10 mM glycine, 150 mM NaCl, pH 3.0, and 10 mM H₂CO₃/HCO₃, and 150 mM NaCl, pH 10.1. Only the BIODYNE® A membrane was used for this study. Membranes not treated with MT washed with PBS containing radioactive cadmium served as the controls. Membranes were placed in the MILLIPORE® filtering unit and processed as follows:
Membrane #1 (blank) was washed with 5 mL of PBS containing radioactive cadmium. The membrane was then washed twice with 10 mL of non-radioactive, metal-free PBS.
Membrane #2 was washed first with 10 mL of MT solution and then 5 mL of PBS containing radioactive cadmium. The membrane was then washed twice with 10 mL of non-radioactive, metal-free PBS.
Membrane #3 was washed with 10 mL of MT solution and then 5 mL of 10 mM H₂CO₃/HCO₃, 150 mM NaCl, pH 10.1, containing radioactive cadmium. The membrane was then washed twice with 10 mL of non-radioactive, metal-free 10 mM H₂CO₃/HCO₃, 150 mM NaCl, pH 10.1.
Membrane #4 was washed with 10 mL of MT solution and then 5 mL of 10 mM glycine, 150 mM NaCl, pH 2.0, containing radioactive cadmium. The membrane was then washed twice with 10 mL of non-radioactive, metal-free 10 mM glycine, 150 mM NaCl, pH 2.0.

Each membrane was analyzed for radioactivity as described above. The results are shown below in Table 3.

TABLE 3

| Sample | CPM |
|---|---|
| Membrane 1 (blank) | 174 |
| Membrane 2 pH 7.5 | 33380 |
| Membrane 3 pH 10.1 | 6890 |
| Membrane 4 pH 2.0 | 651 |

This experiment demonstrates that the membrane-bound MT is capable of binding metal at pHs ranging from 7.5 to 10.1 but does not occur at a pH of 2. Once metal is bound to the MT, it can be recovered by exposing the membrane to acid (pH=2) (See FIG. 4 for a schematic of the procedure). These experiments were conducted by adding all the solutions directly to the membrane. To evaluate effects of pre-equilibrating the membranes with buffer prior to addition of MT, i.e., is the efficiency of metal-binding effected, membranes (BIODYNE® B) were processed as follows:
Membrane #1 (blank) was washed with 5 mL of PBS containing radioactive cadmium. The membrane was then washed twice with 10 mL of non-radioactive, metal-free PBS.
Membrane #2 was washed first with 10 mL of MT solution and then 5 mL of PBS containing radioactive cadmium. The membrane was then washed twice with 10 mL of non-radioactive, metal-free PBS.
Membrane #3 was pre-washed with 10 mL of metal-free 10 mM H₂CO₃/HCO₃, 150 mM NaCl, pH 10.1, then washed with 10 mL of MT solution and then 5 mL of 10 mM H₂CO₃/HCO₃, 150 mM NaCl, pH 10.1, containing radioactive cadmium. Finally, the membrane was washed twice with 10 mL of non-radioactive, metal-free 10 mM H₂CO₃/HCO₃, 150 mM NaCl, pH 10.1.

The results are shown below in Table 4.

TABLE 4

| Sample | CPM |
|---|---|
| Membrane #1 | 190 |
| Membrane #2 | 4218 |
| Membrane #3 | 7431 |

Equilibrating the membrane at pH 10.1 results in better efficiency of protein binding to the membrane.

### Specificity of MT Metal-Binding

Binding affinity/specificity was measured against bovine serum albumin, a protein containing several cysteine residues and known to bind heavy metals. The BIODYNE® A membrane was used for this experiment. The concentration of MT solution was found to be approximately 7 μg/mL. The concentration of the flow through is equivalent to the starting material indicating that the amount bound to the membrane is in ng (nanograms), thus indicating that the metal-binding capacity of the protein is significant. Therefore, 7 μg/mL and 100 μg/mL solutions of BSA were made in D-PBS using a 2 mg/mL BSA standard. The cadmium binding solution was prepared as follows: 1.5 mL of aqueous 1 ppm CdCl₂ was mixed with 3 μL of ¹⁰⁹Cd. The solution was stored at 4° C. The assay was run as follows:
Membrane #1 (blank) was washed with 5 mL of PBS containing radioactive cadmium. The membrane was then washed twice with 10 mL of non-radioactive, metal free PBS.
Membrane #2 was washed first with 5 mL of MT solution and then 5 mL of PBS containing radioactive cadmium. The membrane was then washed twice with 10 mL of non-radioactive, metal free PBS.
Membrane #3 was washed with 5 mL of BSA solution (7 μg/mL) and then 5 mL of PBS containing radioactive cadmium. The membrane was then washed twice with 10 mL of non-radioactive, metal-free PBS.
Membrane #4 was washed with 10 mL of BSA solution (100 μg/mL) and then 5 mL of PBS containing radioactive cadmium. The membrane was then washed twice with 10 mL of non-radioactive, metal-free PBS.

The results of these experiments are shown below in Table 5.

TABLE 5

| Sample | | CPM |
|---|---|---|
| Membrane 1 | No MT | 174 |
| Membrane 2 | MT (5 mL) | 1171 |
| Membrane 3 | BSA (5 mL of 7 μg/mL) | 77 |
| Membrane 4 | BSA (10 mL of 100 μg/mL) | 151* |

*this membrane was tested a different day where the MT binding activity was greater than 3000 CPM.

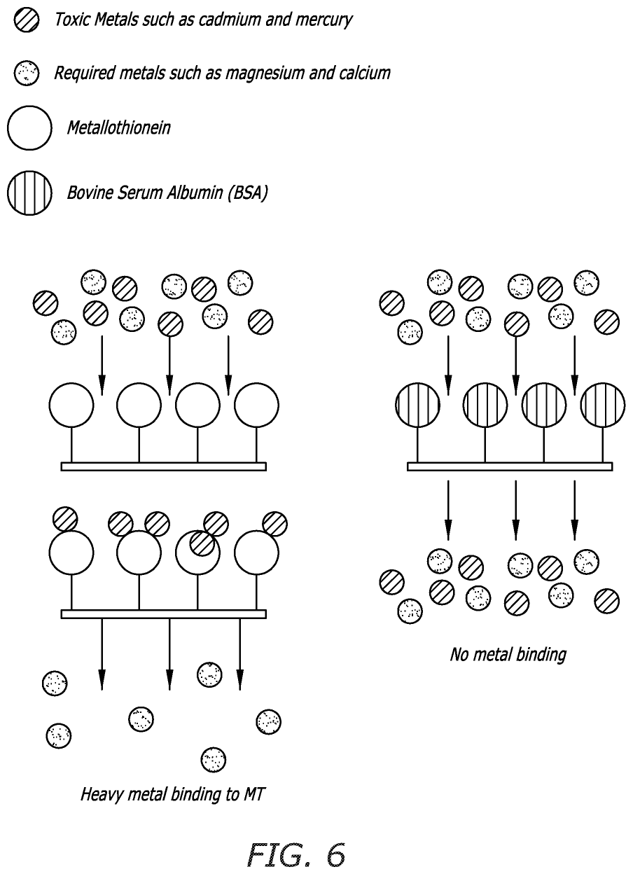

Under these experimental conditions, BSA does not remove metal from aqueous solutions, even when using a 10-fold higher concentration of BSA than MT to prepare the membrane. This experiment demonstrates the utility of membrane bound MT for remediation of metal from water or other aqueous substrates (See FIG. 6 for a schematic of the procedure).

### Effect of Temperature on Metal-Binding Activity.

These binding experiments were performed with BIODYNE® A membranes.
Membrane #1 (blank) was washed with 5 mL of PBS containing radioactive cadmium. The membrane was then washed twice with 10 mL of non-radioactive, metal-free PBS.
Membrane #2 was washed with 10 mL of MT solution and then 5 mL of PBS containing radioactive cadmium pre-warmed to 60° C. The membrane was then washed twice with 10 mL of non-radioactive, metal-free PBS pre-warmed to 60° C.
Membrane #3 was washed with 10 mL of MT solution and then 5 mL of PBS containing radioactive cadmium cooled to 4° C. The membrane was then washed twice with 10 mL of non-radioactive, metal free PBS cooled to 4° C.

The results of these experiments are shown below in Table 6.

TABLE 6

| Sample | CPM |
|---|---|
| Membrane #1 | 139 |
| Membrane #2 | 3886 |
| Membrane #3 | 2672 |

### Example 6

### Comparison of Rabbit and Artemia MT

Metal remediation with the metal-binding proteins disclosed herein can be accomplished using MT proteins from a variety of sources. Rabbit liver MT was obtained as a lyophilized protein (Sigma) and solubilized in 400 μL of 50 mM Tris, pH 8.0, 0.001 M DTT to a final concentration of 2.5 mg/mL (rabbit MT stock solution). Artemia MT was purified as described supra in Example 4.

Membranes were prepared having bound Artemia MT or rabbit liver MT by passing an MT-containing solution through the membrane, as described supra in Example 4. Three membranes, a blank, a membrane bound with Artemia MT and a membrane bound with rabbit liver MT, were then placed in a 13 mm scintered glass filtering unit and 10 mL of a metal-binding solution (a stock solution of 9000 cpm of ¹⁰⁹Cd/25 μL of solution diluted to 75 μL/10 mL PBS to form the metal-binding solution) was passed through the membrane under vacuum. The membrane was then washed three times in PBS, and the membrane-bound radioactivity was measured in a Packard gamma counter. In a second experiment, a larger quantity of Artemia MT was bound to the membrane. The results of these two experiments are found in Tables 7 and 8.

TABLE 7

| Sample | | CPM |
|---|---|---|
| Membrane 1 | Blank | 351 |
| Membrane 2 | Artemia (20 mL bound to the | 685 |
| | membrane | |
| Membrane 3 | Rabbit (25 μL of a 2.5 mg/mL | 985 |
| | solution | |

TABLE 8

| | Sample | CPM |
|---|---|---|
| Membrane 1 | Blank | 231 |
| Membrane 2 | Artemia (25 mL bound to the | 980 |
| | membrane | |

Membrane-bound MT, regardless of source, provides removal of metals from aqueous solutions. In addition, the metal-binding activity is a function of the amount of protein applied to the membrane and increasing the amount of MT protein on the membrane results in increased metal-binding activity by the membrane.

### Example 7

### Expression and Purification of Recombinant Chimeric MT Protein

An MT N-his-tagged SUMO (small ubiquitin-like modifier) chimeric protein was produced and expressed using an E. coli inducible T7 expression system. The chimeric protein retains its metal-binding activity.

### Cloning of N-His-Tagged SUMO MT Fusion Construct

The forward primer incorporated the 5′ end of the MT coding sequence and an 18 bp overlap corresponding to the C-terminus of the SUMO protein. The reverse primer included the 3′ end of the MT coding sequence and an 18 bp overlap containing a stop codon and vector sequence. PCR amplification of 5 ng of MT coding sequence produced a 180 bp product. The PCR product was gel purified. To produce SUMO-MT clones, ligation independent cloning was performed by incubating 25 ng of N-his-SUMO-pETite vector (Lucigen), 59 ng of purified PCR product and 40 μL HI-Control 10G chemically competent cells (Lucigen) on ice for 30 min. The mixture was heat-shocked at 42° C. for 45 sec followed by 2 min on ice. Cells were incubated with 960 μL recovery medium at 37° C. for 1 hr. Cells were plated on YT agar containing 30 μg/mL kanamycin and 1% glucose. Clones were selected and verified for the SUMO-MT insert by PCR screening. Plasmid DNA was purified using QIAGEN® midi columns. The SUMO-MT sequence was verified by DNA sequencing. To establish SUMO-MT expression clones, 6 ng of purified plasmid was transformed into HI-Control BL21(DE3) chemically competent cells (Lucigen) as described previously. Confirmation of insert and SUMO-MT sequence was verified as previously described. The construct was referred to as pCODAₛᵤₘₒ₋ₘₜ.

(SEQ ID NO: 2)
HHHHHHGSLQDSEVNQEAKPEVKPEVKPETHINLKVSDGSSEIFFKIKKT
TPLRRLMEAFAKRQGKEMDSLTFLYDGIEIQADQTPEDLDMEDNDIIEAH
REQIGGMDCCKNGCTCAPNCKCAKDCKCCKGCECKSNPECKCEKNCSCNS
CGCH

### Expression of MT and N-His-Tagged SUMO-MT Using Shake-Flask Technique

The MT gene was cloned into pET 11a. The construct is referred to as pCODAₘₜ. Recombinant MT was expressed in BL-21 E. coli. LB Broth was inoculated with either pCODAₘₜ or pCODAₛᵤₘₒ₋ₘₜ and incubated overnight at 37° C. and 220 rpm. The following morning, a one liter LB culture was inoculated with 14 mL of the overnight culture. The cells were cultured to an A₅₆₀ between 0.4-0.6. IPTG was added to the culture to a final concentration of 1 mM to induce expression of MT.

### Expression and Purification of N-His-Tagged SUMO-MT Using Fermentation Technique

N-his-tagged SUMO-MT was expressed in BL-21 E. coli. LB broth was inoculated with pCODAₛᵤₘₒ₋ₘₜ and incubated overnight at 37° C. and 220 rpm. Next morning, 2.5 L of fermentation media was inoculated with 125 mL of overnight culture. The culture was maintained at 37° C. and pH 7.0. After the nutrition was exhausted, 25 g of glucose and 0.5 g of ammonium chloride was added. The cells were cultured to an A₆₀₀ of 15. IPTG was added to the culture to a final concentration of 1 mM to induce expression of MT. Temperature was lowered to 20° C. for a 3 hr induction period.

### Purification of N-His-Tagged SUMO-MT Using Affinity Chromatography

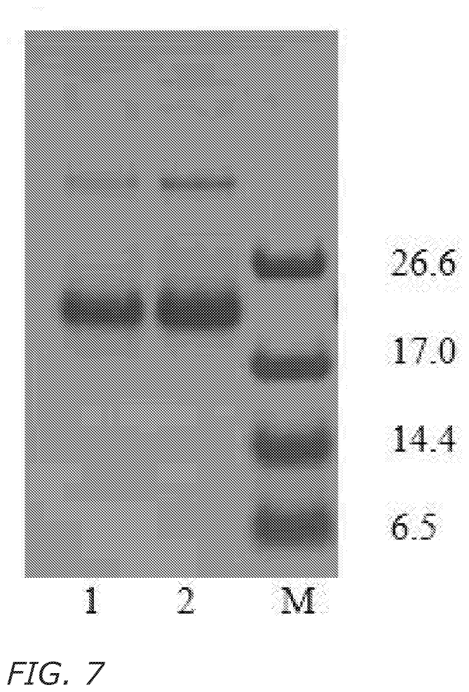

Cells were collected and lysed by sonication in 50 mM Na₂HPO₄, 300 mM NaCl, pH 8.0. The lysate was placed in boiling water for 10 min then clarified by centrifugation. Five to ten milligrams protein per milliliter Ni-NTA agarose (resin) was mixed. The mixture was incubated in 50 mM Na₂HPO₄, 300 mM NaCl, pH 8.0, overnight at 4° C. The resin was washed with 50 mM Na₂HPO₄, 500 mM NaCl, 10 mM imidazole, pH 8.0. N-his-tagged SUMO-MT was eluted with 400 mM imidazole. Protein concentration was determined and the purity of the preparation determined by SDS-PAGE 12% Bis-Tris gel (FIG. 7).

### Example 8

### Mass Spectrometry Sample Preparation

Eluted fractions were analyzed by SDS-PAGE on a NuPAGE 12% Bis-Tris gel (Life Technologies). The His-SUMO-MT protein has a molecular weight of 17.3 kDa but appears as a large 22 kDa band.

The 22 kDa band was verified as the His-SUMO-MT protein by mass spectrometry. Sample preparation for mass spectrometry analysis involved in gel digestion and zip-tip cleanup. The 22 kDa band was excised for the gel and cut into small pieces. The gel pieces were destained with 100 μl 1:1 (v/v) 50% CH₃CN and 25 mM NH₄CO₃, incubated on a rotary shaker for 10 min at room temperature, centrifuged at 13,000 rpm for 1 min, and the supernatant removed. This process was repeated twice more. For sample desalting 100 μl of mass spectrometry grade water was added to the gel pieces, followed by incubation on a rotary shaker for 10 min at room temperature, centrifuged at 13,000 rpm for 1 min, and the supernatant removed. This process was repeated twice more. To dry the gel, 100 μl 100% CH₃CN was added. The sample was incubated on a rotary shaker for 10 min at room temperature. The supernatant was discarded and the sample allowed to air dry for 15 min. For reduction of the protein, the sample was rehydrated in 100 μl 25 mM DTT in 25 mM NH₄CO₃ and incubated at 56° C. for 20 min. The sample was cooled to room temperature and the residual liquid removed. Proteins were alkylated with 100 μl 55 mM iodoacetamide in 25 mM NH₄CO₃ and incubated at room temperature in the dark. The gel pieces were dehydrated for 5 min in 200 μl 25 mM NH₄CO₃/50% CH₃CN followed by 30 sec in 100% CH₃CN. Gel particles were vacuum-dried for 2 min, and resuspended in 32 μl mass spectrometry grade water. Endoproteinase digestion was with 8 μl 5× digestion buffer (250 mM Tris-HCl in 2.5 mM ZnC₄H₆O₄, pH 8.0, Thermo Scientific) and Aspartate-N enzyme (Thermo Scientific) at a ratio of 1 volume of enzyme per 20 volumes of protein. The samples were digested at 37° C. for 24 hr. The digest was centrifuged at 13,000 rpm for 1 min and the liquid collected. Seventy-five microliters of 1% trifluoroacetic acid (TFA)/70% CH3CN was added to the digest tube, incubated on a rotary shaker for 20 min at room temperature, and centrifuged for 13,000 rpm for 1 min. The liquid was pooled with the liquid collected previously. The process was repeated. The pooled liquid was incubated on dry ice for 10 sec and vacuum dried for 80 min.

ZIPTIP® (Merck, Darmstadt, Germany) clean up was then performed on the digested protein product as follows. The ZIPTIP® was wetted and equilibrated with 100% CH₃CN followed by 0.1% TFA. The sample was resuspended in 20 μl 0.1% TFA and centrifuged for 1 min at 13,000 rpm. The protein sample was applied to the tip, and the tip was washed several times with 0.1% TFA. The sample was eluted with 0.1% TFA and 50% acetonitrile, and vacuum-dried for 30 min. The SUMO and MT sequence was verified by MOLDI-TOF mass spectroscopy.

### Example 9

### Determination of Metal-Binding Activity of SUMO-MT

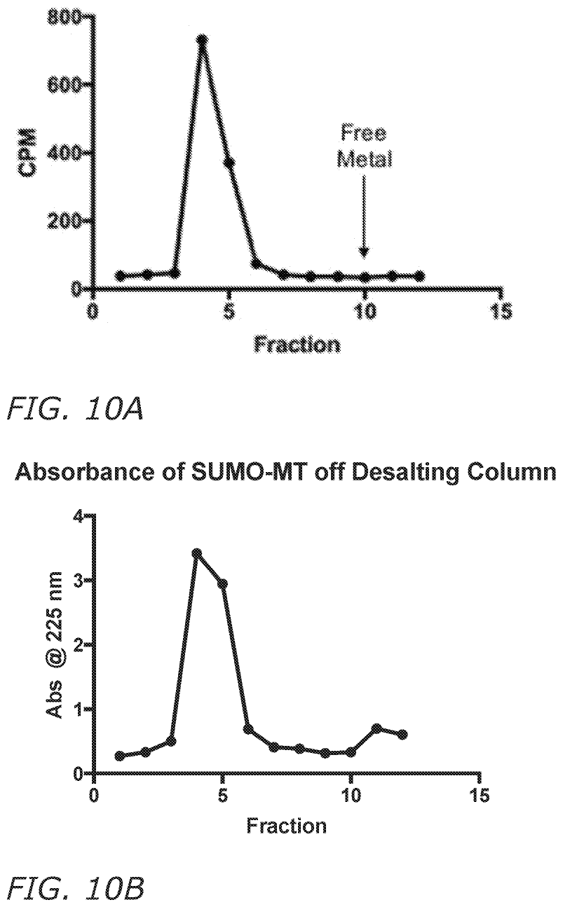

The metal binding activity of the SUMO-MT was determined as follows: A 5 ml D-Salt-Excellulose Desalting column (Pierce Chemical Company) was equilibrated with three bed volumes (15 ml) of PBS (0.01 M Phosphate, 0.9% (w/v) NaCl, pH 7.4. The PBS was allowed to completely enter the column. Four hundred microliters of a 1 mg/ml solution of SUMO-MT was mixed with 10 microliters of an aqueous solution of metal (2 mg/10 ml). The mixture was applied to the surface of the desalting column and allowed to completely enter the column. Then 500 μl of PBS was added to the column and the eluate collected. This procedure was repeated 11 more times generating twelve individual 500 μl fractions. Fractions were analyzed for metal using ICPMS. A typical elution profile using ¹⁰⁹Cd is shown in FIGS. 10A and 10C. The level of ¹⁰⁹Cd in each fraction was determined using liquid scintillation. The elution profile of the SUMO-MT without added metal was determined by applying the protein to the column and measuring the A₂₂₅ of the fractions (FIGS. 10B and 10C).

### Example 10

### Removal of Metals from Organic Solvents

Purified SUMO-MT was used to remove palladium nanoparticles from chloroform. The palladium nanoparticles were dissolved in chloroform to a final concentration of 4 mg/ml. The λₘₐₓ of the solution was 487 nm. One and a half ml aliquots of the solution were transferred to each of two other 15 ml conical centrifuge tubes. Then one ml of a 2 mg/ml solution of SUMO-MT in Tris, pH 8.0, 15% (w/v) trehalose, and 0.1 mM DTT was transferred to each of the two tubes containing the copper solution. The mixture was shaken vigorously and centrifuged at 1000 rpm and 4° C. for one min. The A₄₈₇ of the lower organic phase was measured to determine the amount of remaining nanoparticles. The efficiency of removal of the nanoparticles from the chloroform was calculated to be 80%.

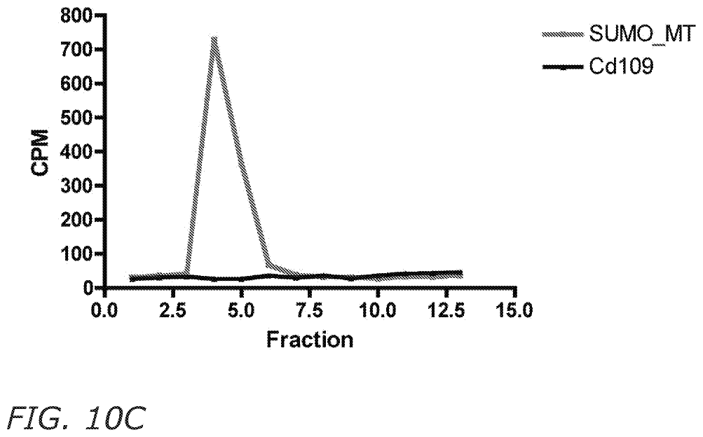

As shown in the FIG. 11, the Pd nanoparticles were removed from the chloroform using SUMO-MT.

Purified SUMO-MT was used to remove gold nanoparticles from chloroform. The gold nanoparticles were dissolved in chloroform to a final concentration of 0.25 mg/ml. The λₘₐₓ of the solution was 324 nm. Two and a half ml aliquots of the solution were transferred to each of two other 15 ml conical centrifuge tubes. Then one and a half ml of a 2 mg/ml solution of SUMO-MT in Tris, pH 8.0, 15% (w/v) threalose and 0.1 mM DTT was transferred to each of the two tubes containing the gold nanoparticles. The mixture was shaken vigorously and centrifuged at 1000 rpm and 4° C. for one min. The A₃₂₄ of the lower organic phase was measured to determine the amount of remaining nanoparticles. The efficiency of removal of the nanoparticles from the chloroform was calculated to be 55%.

Purified SUMO-MT was used to remove complex chloro [1,3 bis(2,6-diisopropylphenyl)imidazole-2-ylidene]copper(I) from chloroform. The compound is insoluble in water.

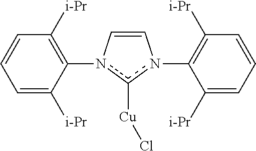

The copper complex was dissolved in chloroform to a final concentration of 4 mg/ml. The λₘₐₓ of the solution was 344 nm. One and a half ml aliquots of the copper solution were transferred to each of two other 15 ml conical centrifuge tubes. Then one ml of a 2 mg/ml solution of SUMO-MT in Tris buffer, pH 8.0, was transferred to each of the two tubes containing the copper solution. The mixture was shaken vigorously and centrifuged at 1000 rpm and 4° C. for one min. The A₃₄₄ of the lower organic phase was measured to determine the amount of remaining copper complex. The efficiency of removal of the copper complex from the chloroform was calculated to be 64%.

### Example 11

### Removal of Radioactive Metals from Organic Solvents

Purified SUMO-MT was used to remove uranium from 30% (v/v) tributyl phosphate in turpentine. The tributyl phosphate is first used to extract uranium from acid solutions. This is the same method as uranium can be recovered from spent nuclear fuel rods. Uranyl acetate was solubilized in 1 M HNO₃ to a final concentration of 100 mM. Uranyl acetate solution was mixed with an equal volume of 30% (v/v) tributyl phosphate in turpentine. The solution was mixed well and allowed to sit at room temperature for 5 min. The mixture was centrifuged at 1000 rpm for 1 min at 4° C. Two and a half ml of the upper organic phase was transferred to another centrifuge tube and mixed with 4.5 ml of 50 mM Tris, pH 8.0. The mixture was centrifuged at 1000 rpm for 1 min at 4° C. The upper organic phase was washed a second time with 50 mM Tris, pH 8.0 to be sure the pH is above 4. Two ml of the washed organic phase was mixed with 1 ml of SUMO-MT linked to an agarose resin. The mixture was mixed well and centrifuged at 1000 rpm for 1 min at 4° C. The organic phase was analyzed for uranium using ICPMS. Under these conditions, the calculated removal of the uranium from the organic phase was 67% efficient.

### Example 12

### Removal of Metal (¹⁰⁹Cd) from Sea Water Using Membrane Bound SUMO-MT

A PALL BIODYNE® membrane (Biodyne A 0.45 μm) was used as a solid support for these experiments. A 1 cm² piece of membrane was placed in a 10 mL MILLIPORE® glass frit filtering unit. Five milliliters of SUMO-MT (0.2 mg/ml in 50 mM Tris, pH 8.0) was passed through the membrane under vacuum at a flow rate of approximately 50 mL/min. Next, 10 mL of a solution of cadmium (0.2 ng of ¹⁰⁹Cd in 30 mL of sea water) was passed through the membrane under vacuum. The membranes was then washed twice, each with 10 mL of PBS. The membrane was removed from the filtering unit, and analyzed for radioactivity. As a control, the procedure was repeated with a second membrane not treated with MT. This membrane is referred to as the “blank.” The results are shown below in Table 9.

TABLE 9

| | Sample | CPM |
|---|---|---|
| Membrane 1 | Blank | 118 |
| Membrane 2 | +SUMO-MT | 18318 |

Removal of Metal (Cu) from Water Using SUMO-MT Bound to Agarose Beads

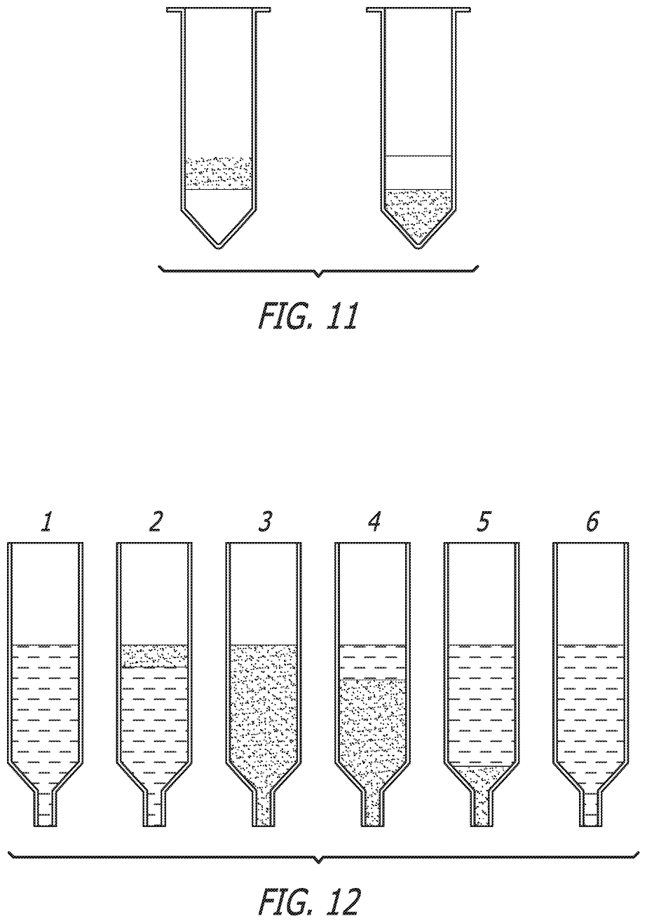

Purified SUMO-MT was covalently linked to agarose beads. Two ml of the material was loaded into a small chromatography column and washed with 50 mM tris, pH8.0. A 0.1 mM solution of copper sulfate was pumped through the column at a flow rate of 0.5 ml/min. Binding of the metal to the column was evident by the change in color (See FIG. 12). The metal did not bind to a column of agarose beads without MT. The metal was recovered from the resin by flushing the column with 1.0 ml of 1.0 M HCl. The metal binding activity of the column was regenerated by washing the column with water then with 50 mM Tris, pH8.0. The column remains active after four regeneration cycles.

### Effect of pH on Metal Binding of Recombinant SUMO-MT

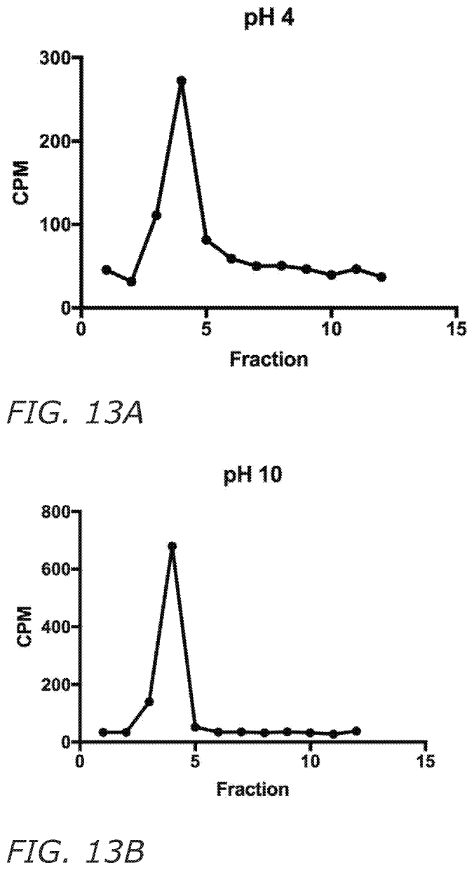

Two D-Salt Excellulose desalting columns were equilibrated with buffers of different pH. One with acetate buffer pH, 4.0 and the other bicarbonate buffer, pH 10.0. Then 0.02 μg of ¹⁰⁹Cd was mixed with 400 μl of SUMO-MT (1 mg/ml). The mixture was applied to the surface and allowed to completely enter the column. Then 500 μl of the appropriate buffer was added to the column and the eluate collected. This procedure was repeated 11 times generating twelve individual 500 μl fractions. The fractions were analyzed for radioactivity by liquid scintillation. The metal binding activity at both pH's is shown in FIGS. 13A and B.

In closing it is to be understood that the embodiments of the invention disclosed herein are illustrative of the principals of the invention. Other modifications may be employed which are within the scope of the invention and accordingly, the present invention is not limited to that precisely as shown and described in the present specification.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A recombinant chimeric metallothionein (MT) protein having a sequence at least 90% identical to full-length SEQ ID NO:1.

2. A device for removing heavy metals from a substrate comprising:
a regenerative metal-binding material comprising a MT protein having a sequence at least 90% identical to one of SEQ ID Nos. 1, 2, and 9-20;
wherein the regenerative metal-binding material binds heavy metals thereby removing the heavy metals from a substrate; and
wherein the binding of heavy metal to the regenerative metal-binding material is reversible and wherein the regenerative metal-binding material is reusable.

3. The device according to claim 2, wherein the regenerative metal-binding material comprises the MT protein in an aqueous solution.

4. The device according to claim 2, wherein the regenerative metal-binding material comprises the MT protein associated with a solid support.

5. The device according to claim 4, wherein the solid support is a resin.

6. The device according to claim 4, wherein the solid support is a membrane.

7. The device according to claim 2, wherein the substrate is a metal-containing liquid.

8. The device according to claim 7 wherein the substrate is a metal-containing organic liquid.

9. The device according to claim 2, wherein the heavy metal is a heavy metal complex.

10. The device according to of claim 2, wherein the metal is substantially released from the metal-binding protein at a pH of about 1.

11. The device according to claim 2, wherein the metal-binding protein has a substantial metal-binding affinity at a pH above about pH 5.

12. A method for removing metals from a substrate comprising:
contacting a substrate having heavy metals therein with a metal-binding material comprising a MT protein having a sequence at least 90% identical to one of SEQ ID NO:1, 2, and 9-20;
binding the heavy metal to the metal-binding material thereby producing a substrate having less heavy metal contained therein.

13. The method according to claim 12, wherein the metal-binding material comprises the MT protein in an aqueous solution.

14. The method according to claim 12, wherein the metal-binding material comprises the MT protein associated with a solid support.

15. The method according to claim 14, wherein the solid support is a resin.

16. The method according to claim 14, wherein the solid support is a membrane.

17. The method according to claim 12, wherein the heavy metal is a heavy metal complex.

18. The method according to claim 12, wherein the substrate is a metal-containing liquid.

19. The method according to claim 18, wherein the substrate is a metal-containing organic liquid.

20. The method according to claim 12, further comprising:
releasing the bound heavy metal from the metal-binding material; and
regenerating the metal-binding capacity of the metal-binding material

21. The method of claim 12, wherein the metal is substantially released from the metal-binding protein at a pH of about 1.

22. The method of claim 12, wherein the metal-binding protein has a substantial metal-binding affinity at a pH above about pH 5.

23. An aqueous solution for extracting a radioactive metal from an organic solvent, the aqueous solution comprising a metal-binding protein having an amino acid sequence at least 90% identical to one of SEQ ID NO:1, 2, or 9-20.

24. The aqueous solution of claim 23, wherein the metal is a heavy metal, a radioactive metal, or a precious metal.

25. The aqueous solution of claim 23, wherein the metal is a transition metal.

26. The aqueous solution of any one of claims 23-25, wherein the metal has an atomic weight greater than 40 g/mol.

27. The aqueous solution of claim 23, wherein the organic solvent comprises dichloromethane, chloroform, diethyl ether, or hexane.

28. The aqueous solution of claim 27, wherein the organic solvent comprises chloroform.

29. The aqueous solution of claim 27, wherein the organic solvent comprises dichloromethane.

30. The aqueous solution of claim 27, wherein the organic solvent comprises diethyl ether.

31. The aqueous solution of claim 27, wherein the organic solvent comprises hexane.

32. The aqueous solution of claim 23, wherein the metal is substantially released from the metal-binding protein at a pH of about 1.

33. The aqueous solution of claim 23, wherein the metal-binding protein has a substantial metal-binding affinity at a pH above about pH 5.
